(19) United States
(12) Patent Application Publication
(10) Pub. No.: US 2011/0319280 A1
(43) Pub. Date: Dec. 29, 2011

(54) ECT2 ONCOGENE AS A THERAPEUTIC TARGET AND PROGNOSTIC INDICATOR FOR LUNG AND ESOPHAGEAL CANCER

(75) Inventors: Yusuke Nakamura, Tokyo (JP); Yataro Daigo, Tokyo (JP); Akira Togashi, Kanagawa (JP)

(73) Assignee: Oncotherapy Science, Inc., Kawasaki-shi, Kanagawa (JP)

(21) Appl. No.: 13/002,977

(22) PCT Filed: Jul. 16, 2009
(86) PCT No.: PCT/JP2009/003360

§ 371 Date: Sep. 16, 2011

Related U.S. Application Data
(60) Provisional application No. 61/081,165, filed on Jul. 16, 2008.

(51) Int. Cl. C40B 30/04; G01N 33/566; C12Q 1/68; G01N 33/53

(52) U.S. Cl. 506/9; 436/501; 435/6.11; 435/6.12; 435/7.9

(57) ABSTRACT

The invention features methods for detecting lung cancer or esophageal cancer, by detecting over-expression of ECT2 compared the normal organs. Also disclosed are methods of identifying compounds for treating and preventing lung cancer or esophageal cancer, based on the over-expression of ECT2 in the lung cancer or esophageal cancer, the cell proliferation function of ECT2. Also, provided are a method for treating lung cancer or esophageal cancer by administering a double-stranded molecule against the ECT2 gene or an antibody against ECT2 protein. The invention also provides products, including the double-stranded molecules and vectors encoding them, as well as compositions comprising the molecules or vectors, useful in the provided methods.

### CROSS-REFERENCE TO RELATED APPLICATIONS

The present application claims the benefit of U.S. Provisional Application No. 61/081,165, filed Jul. 16, 2008, the entire disclosure of which is hereby incorporated herein by reference.

### TECHNICAL FIELD

The present invention relates to methods for detecting, diagnosing, and prognosing cancer as well as methods for treating and preventing cancer.

### BACKGROUND ART

Primary lung cancer is the leading cause of cancer deaths in most countries (Alberg A J. et al. J Clin Oncol 2005; 14:3175-85, Parkin D M. Lancet Oncol 2001; 2:533-43.). Meanwhile esophageal squamous-cell carcinoma (ESCC) is one of the most common fetal malignancies of the digestive tract (Shimada H. et al. Surgery 2003; 133:486-94.). In spite of improvements in surgical techniques and adjuvant chemoradiotherapy, patients with advanced lung or esophageal cancer often suffer fatal disease progression (Parkin D M. Lancet Oncol 2001; 2:533-43, Shimada H. et al. Surgery 2003; 133:486-94.). Therefore, it is extremely important to understand the biology of these two major thoracic cancers, and to introduce more effective treatments in order to improve the survival of patients (Daigo Y. and Nakamura Y. Gen Thorac Cardiovasc Surg 2008; 56:43-53.). The concept of specific molecular targeting has been applied to the development of innovative cancer-treatment strategies, and two main approaches are available at present in clinical practice: therapeutic monoclonal antibodies and small-molecule agents (Thatcher N. Lung Cancer 2007; 57 Suppl 2:S18-23.). To date, four targeted therapies (bevacizumab, cetuximab, erlotinib and gefitinib) have been investigated in randomised trials for the treatment of advanced non-small cell lung cancer (NSCLC) (Thatcher N. Lung Cancer 2007; 57 Suppl 2:S18-23, Sandler A. et al. N Engl J Med 2006; 355:2542-50, Shepherd F A. et al. N Engl J Med 2005; 353:123-32, Thatcher N. et al. Lancet 2005; 366:1527-37.). The addition of therapeutic antibodies against pro-angiogenic protein vascular endothelial growth factor (VEGF) (bevacizumab) or epidermal growth factor receptor (EGFR) (cetuximab) to conventional chemotherapy has a significant survival benefit in patients with NSCLC (Thatcher N. Lung Cancer 2007; 57 Suppl 2:S18-23, Sandler A. et al. N Engl J Med 2006; 355:2542-50.). Two small-molecule EGFR tyrosine kinase inhibitors, erlotinib and gefitinib, were shown to be effective for a subset of advanced NSCLC patients (Shepherd F A. et al. N Engl J Med 2005; 353:123-32, Thatcher N. et al. Lancet 2005; 366:1527-37.). However, issues of toxicity limit these treatment regimens to selected patients, and even if all available treatments are applied, the proportion of patients showing good response is still very limited (Thatcher N. Lung Cancer 2007; 57 Suppl 2:S18-23, Sandler A. et al. N Engl J Med 2006; 355:2542-50, Shepherd F A. et al. N Engl J Med 2005; 353:123-32, Thatcher N. et al. Lancet 2005; 366:1527-37.).

To isolate potential molecular targets for diagnosis, treatment, and/or prevention of lung and esophageal carcinomas, the present inventors previously performed a genome-wide analysis of gene expression profiles of cancer cells from 101 lung-cancer and 19 ESCC patients by means of a cDNA microarray consisting of 27,648 genes or ESTs (Daigo Y. and Nakamura Y. Gen Thorac Cardiovasc Surg 2008; 56:43-53, Kikuchi T. et al. Oncogene 2003; 22:2192-205, Kakiuchi S. et al. Mol Cancer Res 2003; 1:485-99, Kakiuchi S. et al. Hum Mol Genet. 2004; 13:3029-43, Kikuchi T. et al. Int J Oncol 2006; 28:799-805, Taniwaki M. et al. Int J Oncol 2006; 29:567-75, Yamabuki T. et al. Int J Oncol 2006; 28:1375-84.). To verify the biological and clinicopathological significance of the respective gene products, the present inventors have established a screening system by a combination of the tumor-tissue microarray analysis of clinical lung- and esophageal-cancer materials with RNA interference (RNAi) technique (Suzuki C. et al. Cancer Res 2003; 63:7038-41, Ishikawa N. et al. Clin Cancer Res 2004; 10:8363-70, Kato T. et al. Cancer Res 2005; 65:5638-46, Furukawa C. et al. Cancer Res 2005; 65:7102-10, Ishikawa N. et al. Cancer Res 2005; 65:9176-84, Suzuki C. et al. Cancer Res 2005; 65:11314-25, Ishikawa N. et al. Cancer Sci 2006; 97:737-45, Takahashi K. et al. Cancer Res 2006; 66:9408-19, Hayama S. et al. Cancer Res 2006; 66:10339-48, Kato T. et al. Clin Cancer Res 2007; 13:434-42, Suzuki C. et al. Mol Cancer Ther 2007; 6:542-51, Yamabuki T. Cancer Res 2007; 67:2517-25, Hayama S. et al. Cancer Res 2007; 67:4113-22, Kato T. et al. Cancer Res 2007; 67:8544-53, Taniwaki M. et al. Clin Cancer Res 2007; 13:6624-31, Ishikawa N. et al. Cancer Res 2007; 67:11601-11, Mano Y. et al. Cancer Sci 2007; 98:1902-13, Suda T. et al. Cancer Sci 2007; 98:1803-8, Kato T. et al. Clin Cancer Res 2008; 14:2363-70.). In this process, the present inventors identified epithelial cell transforming sequence 2 (ECT2) oncogene as a prognostic biomarker as well as a therapeutic target for lung and esophageal cancers (WO 2004/031413, WO2007/013671).

ECT2 was isolated through an expression cloning strategy from a mouse epithelial cell line BALB/MK, which conferred in vitro transforming activity (Miki T. Methods Enzymol 1995; 256:90-8.). ECT2 is a member of the Dbl family that possesses a Dbl homology (DH)/pleckstrin homology (PH) cassette in the C-terminal end of the protein and mediates the guanine nucleotide exchange of Rho GTPases (Tatsumoto T. et al. J Cell Biol 1999; 1475:921-8.). The N-terminus of ECT2 contains tandem repeats of the BRCT domain, which is conserved in many proteins involved in cell cycle check point and DNA damage response (Kim J E. et al. J Biol Chem 2005; 280:5733-9.). ECT2 is localized at the central spindle and equatorial cortex, and triggers cytokinesis by activating RhoA (Petronczki M. et al. Dev Cell 2007; 12:713-25, Scoumanne A. and Chen X. Cancer Res 2006; 66: 6271-9, Eguchi T. et al. Oncogene 2007; 26:509-20, Hara T. et al. Oncogene 2006; 25:566-78.). ECT2 was indicated to be overexpressed in glioma cells (Sano M. et al. Oncol Rep 2006; 16:1093-8.). In spite of the evidence of ECT2 function in cytokinesis, the significance of activation of ECT2 in human cancer progression and its clinical potential as a therapeutic target have not been not fully described in the prior art.

The present inventors report here the identification of ECT2 as a predictive cancer biomarker in the clinic, and as a useful therapeutic target for pulmonary and esophageal cancer, and also describe the biological roles of ECT2 in progression of cancer.

In recent years, a new approach of cancer therapy using gene-specific siRNA has been used in clinical trials (Bumcrot D et al., Nat Chem Biol 2006 December, 2(12): 711-9). RNAi has earned a place among the major technology platforms (Putral L N et al., Drug News Perspect 2006 July-August, 19(6): 317-24; Frantz S, Nat Rev Drug Discov 2006 July, 5(7): 528-9; Dykxhoorn D M et al., Gene Ther 2006 March, 13(6): 541-52). Nevertheless, improved double-stranded molecules useful for targeting cancer-specific genes are needed for the development of anticancer drugs. The present invention provides such improvements.

### SUMMARY OF INVENTION

The present invention is based on the discovery of a specific expression of the ECT2 gene in cancerous cells.

Through an analysis on genome-wide expression profiles of genes in various types of lung cancer cells, esophageal carcinomas and bladder cancer cells, a set of genes whose expression was commonly up-regulated was identified. From among the genes, the present inventors selected gene ECT2 (epithelial cell transforming sequence 2) for further study. The expression of the ECT2 gene was detected by the present inventors to be enhanced in lung, esophageal and bladder carcinomas. In the course of the present invention, the ECT2 gene was further revealed to be frequently up-regulated in non-small cell lung cancer (NSCLC), including adenocarcinomas (ADCs) and squamous-cell carcinomas (SCCs), small-cell lung cancer (SCLC), and esophageal squamous-cell carcinomas (ESCCs). Furthermore, as shown here for the first time, the suppression of the ECT2 gene by small interfering RNA (siRNA) results in growth inhibition and/or cell death of lung cancer cells. Thus, this gene can now be used as a novel therapeutic target for various types of human neoplasms.

The ECT2 gene identified herein, as well as its transcription and translation products, finds diagnostic utility as a marker for cancer and as an oncogene target, the expression and/or activity of which may be altered to treat or alleviate a symptom of cancer.

Herein, evidence is presented that ECT2 over-expression is associated with lung cancer and ESCC progression, resulting in a poor prognosis for patients with lung cancer and ESCC. Thus, the ECT2 gene is a useful prognostic indicator of lung cancer or ESCC. In particular, ECT2 over-expression in resected specimens is a useful index for application of adjuvant therapy to the patients who are likely to have poor prognosis. Furthermore, in that up-regulation of ECT2 is a frequent and important feature of lung and esophageal carcinogenesis, targeting the ECT2 molecule is particularly useful for development of new diagnostic and therapeutic strategies for clinical management of lung cancers and ESCC.

Accordingly, the present invention provides methods for assessing or determining the prognosis of a patient with lung cancer or esophageal squamous-cell carcinomas by comparing an ECT2 level in a patient-derived biological sample with that of a control sample. An elevated expression level is indicative of a poor prognosis for post-treatment remission, recovery and/or survival and a higher likelihood of poor clinical outcome. The present invention further provides kits for assessing an NSCLC or ESCC prognosis, such kits including ECT2-detection reagents.

Therapeutic methods of the present invention include methods for treating or preventing cancer in a subject including the step of administering an antisense composition to the subject. In the context of the present invention, the antisense composition reduces the expressions of a specific target gene (i.e., the ECT2 gene). For example, the antisense compositions may contain a nucleotide which is complementary to the ECT2 gene sequence. Alternatively, the present methods may include the step of administering an siRNA composition to the subject. In the context of the present invention, the siRNA composition reduces the expression of the ECT2 gene. In yet another method, the treatment or prevention of cancer in a subject may be carried out by administering a double-stranded molecule composition to the subject. In the context of the present invention, the nucleic acid-specific double-stranded molecule composition reduces the expression of the ECT2 gene. In fact, the present inventors have demonstrated the inhibitory effects of siRNAs for the ECT2 gene. For example, the inhibitions of cell proliferation of cancer cells by the siRNAs are demonstrated in the Examples section, which demonstrates that the ECT2 gene serves as a preferable therapeutic target for cancer.

One advantage of the methods described herein is that the disease is identified prior to detection of overt clinical symptoms of cancers. Other features and advantages of the invention will be apparent from the following detailed description, and from the claims. However, it is to be understood that both the foregoing summary of the invention and the following detailed description are of a preferred embodiment, and not restrictive of the invention or other alternate embodiments of the invention.

### BRIEF DESCRIPTION OF DRAWINGS

FIG. 1 depicts the expression of ECT2 in lung and esophageal cancers and normal tissues. A, Expression of ECT2 gene in 15 clinical lung cancers (lung ADC, lung SCC, and SCLC; top panels) and in 15 lung-cancer cell lines (bottom panels), detected by semiquantitative RT-PCR analysis. B, Expression of ECT2 gene in 10 clinical ESCCs and in 10 esophageal cancer cell lines, detected by semiquantitative RT-PCR analysis. C, Expression of ECT2 protein in 6 lung-cancer cell lines and in 4 ESCC cell lines, examined by western-blot analysis. D, Expression of ECT2 gene in normal tissues, detected by northern blotting of mRNAs from 23 normal human tissues (top panel), and ECT2 protein expression examined by immunohistochemical analysis of 5 normal tissues (liver, heart, kidney, lung, and testis) and a lung SCC tissue (bottom panels).

FIG. 2 depicts association of ECT2 overexpression with poor prognosis for NSCLC and ESCC patients. A, the top panels, Representative examples for strong, weak, and absent ECT2 expression in lung SCC tissues and a normal lung tissue (original magnification ×100). The bottom panel, Kaplan-Meier analysis of survival of patients with NSCLC (P=0.0004 by log-rank test). B, the top panels, Representative examples for strong, weak, and absent ECT2 expression in ESCC tissues and a normal esophagus tissue (original magnification ×100). The bottom panel, Kaplan-Meier analysis of survival of patients with ESCC (P=0.0088 by log-rank test).

FIG. 3 depicts inhibition of growth of NSCLC and ESCC cells by siRNAs against ECT2. A, Expression of ECT2 in response to siRNA treatment for ECT2 (si-ECT2-#1 or #2) or control siRNAs (LUC or SCR) in A549 and TE9 cells, analyzed by semi-quantitative RT-PCR (top panels). MTT and colony-formation assays of the tumor cells transfected with si-ECT2s or control siRNAs (middle and bottom panels). B, Flow cytometrical analysis of the A549 cells 48 hours and 72 hours after transfection of the siRNAs for ECT2 (si-ECT2-#1) and control siRNAs (SCR). Transfection of si-ECT2-#1 resulted in G2/M arrest at 48 hours (left panels) and subsequent increase of sub-G1 fraction at 72 hours (right panels).

FIG. 4 depicts enhancement of cellular invasiveness by ECT2 introduction into mammalian cells. The top panels, Transient expression of ECT2 in NIH3T3 and COS-7 cells, detected by western-blot analysis. The middle and bottom panels, assays demonstrating the increased invasive nature of NIH3T3 and COS-7 cells in Matrigel matrix after transfection of ECT2-expressing plasmids. Giemsa staining (magnification, ×100; middle panels) and the number of cells migrating through the Matrigel-coated filters (bottom panels) were shown. Assays were done thrice and in triplicate wells.

### DESCRIPTION OF EMBODIMENTS

### Detailed Description of the Invention

The words “a”, “an”, and “the” as used herein mean “at least one” unless otherwise specifically indicated.

The terms “isolated” and “purified” when used herein in relation to a substance (e.g., polypeptide, antibody, polynucleotide, etc.) indicate that the substance is substantially free from at least one substance that may else be included in the natural source. Thus, an isolated or purified antibody refers to antibodies that is substantially free of cellular material such as carbohydrate, lipid, or other contaminating proteins from the cell or tissue source from which the protein (antibody) is derived, or substantially free of chemical precursors or other chemicals when chemically synthesized. The term “substantially free of cellular material” includes preparations of a polypeptide in which the polypeptide is separated from cellular components of the cells from which it is isolated or recombinantly produced. Thus, a polypeptide that is substantially free of cellular material includes preparations of polypeptide having less than about 30%, 20%, 10%, or 5% (by dry weight) of heterologous protein (also referred to herein as a “contaminating protein”). When the polypeptide is recombinantly produced, it is also preferably substantially free of culture medium, which includes preparations of polypeptide with culture medium less than about 20%, 10%, or 5% of the volume of the protein preparation. When the polypeptide is produced by chemical synthesis, it is preferably substantially free of chemical precursors or other chemicals, which includes preparations of polypeptide with chemical precursors or other chemicals involved in the synthesis of the protein less than about 30%, 20%, 10%, 5% (by dry weight) of the volume of the protein preparation. That a particular protein preparation contains an isolated or purified polypeptide can be shown, for example, by the appearance of a single band following sodium dodecyl sulfate (SDS)-polyacrylamide gel electrophoresis of the protein preparation and Coomassie Brilliant Blue staining or the like of the gel. In a preferred embodiment, antibodies of the present invention are isolated or purified.

An “isolated” or “purified” nucleic acid molecule, such as a cDNA molecule, can be substantially free of other cellular material, or culture medium when produced by re-combinant techniques, or substantially free of chemical precursors or other chemicals when chemically synthesized. In a preferred embodiment, nucleic acid molecules encoding antibodies of the present invention are isolated or purified.

The terms “polypeptide”, “peptide”, and “protein” are used interchangeably herein to refer to a polymer of amino acid residues. The terms apply to amino acid polymers in which one or more amino acid residue is a modified residue, or a non-naturally occurring residue, such as an artificial chemical mimetic of a corresponding naturally occurring amino acid, as well as to naturally occurring amino acid polymers.

The term “amino acid” refers to naturally occurring and synthetic amino acids, as well as amino acid analogs and amino acid mimetics that similarly functions to the naturally occurring amino acids. Naturally occurring amino acids are those encoded by the genetic code, as well as those modified after translation in cells (e.g., hydroxyproline, gamma-carboxyglutamate, and O-phosphoserine). The phrase “amino acid analog” refers to compounds that have the same basic chemical structure (an alpha carbon bound to a hydrogen, a carboxy group, an amino group, and an R group) as a naturally occurring amino acid but have a modified R group or modified backbones (e.g., homoserine, norleucine, methionine, sulfoxide, methionine methyl sulfonium). The phrase “amino acid mimetic” refers to chemical compounds that have different structures but similar functions to general amino acids.

Amino acids may be referred to herein by their commonly known three letter symbols or the one-letter symbols recommended by the IUPAC-IUB Biochemical Nomenclature Commission.

The terms “polynucleotides”, “oligonucleotide”, “nucleotides”, “nucleic acids”, and “nucleic acid molecules” are used interchangeably unless otherwise specifically indicated and, similarly to the amino acids, are referred to by their commonly accepted single-letter codes. Similar to the amino acids, they encompass both naturally-occurring and non-naturally occurring nucleic acid polymers. The polynucleotide, oligonucleotide, nucleotides, nucleic acids, or nucleic acid molecules may be composed of DNA, RNA or a combination thereof.

The present invention is based in part on the discovery of elevated expression of the ECT2 gene in cells from patients of lung and esophageal cancers. The nucleotide sequence of the human ECT2 gene is shown in SEQ ID NO: 11 and is also available as GenBank Accession No. NM_{—}018098. Herein, the ECT2 gene encompasses the human ECT2 gene as well as those of other animals, including non-human primate, mouse, rat, dog, cat, horse, and cow. However, the invention is not limited thereto and includes allelic mutants and genes found in other animals as corresponding to the ECT2 gene.

The amino acid sequence encoded by the human ECT2 gene is shown in SEQ ID NO: 12 and is also available as GenBank Accession No. NP_{—}060568.3. In the present invention, the polypeptide encoded by the ECT2 gene is referred to as “ECT2”, and sometimes as “ECT2 polypeptide” or “ECT2 protein”.

According to an aspect of the present invention, functional equivalents are also considered to be “ECT2 polypeptides”. Herein, a “functional equivalent” of a protein is a polypeptide that has a biological activity equivalent to the protein. Namely, any polypeptide that retains the biological ability of the ECT2 protein may be used as such a functional equivalent in the present invention. Such functional equivalents include those wherein one or more amino acids are substituted, deleted, added, or inserted to the natural occurring amino acid sequence of the ECT2 protein. Alternatively, the polypeptide may be composed an amino acid sequence having at least about 80% homology (also referred to as sequence identity) to the sequence of the respective protein, more preferably at least about 90% to 95% homology. In other embodiments, the polypeptide can be encoded by a polynucleotide that hybridizes under stringent conditions to the natural occurring nucleotide sequence of the ECT2 gene.

A polypeptide of the present invention may have variations in amino acid sequence, molecular weight, isoelectric point, the presence or absence of sugar chains, or form, depending on the cell or host used to produce it or the purification method utilized. Nevertheless, so long as it has a function equivalent to that of the human ECT2 protein of the present invention, it is within the scope of the present invention.

The phrase “stringent (hybridization) conditions” refers to conditions under which a nucleic acid molecule will hybridize to its target sequence, typically in a complex mixture of nucleic acids, but not detectably to other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10 degrees C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times of background, preferably 10 times of background hybridization. Exemplary stringent hybridization conditions include the following: 50% formamide, 5×SSC, and 1% SDS, incubating at 42 degrees C., or, 5×SSC, 1% SDS, incubating at 65 degrees C., with wash in 0.2×SSC, and 0.1% SDS at 50 degrees C.

In the context of the present invention, a condition of hybridization for isolating a DNA encoding a polypeptide functionally equivalent to the human ECT2 protein can be routinely selected by a person skilled in the art. For example, hybridization may be performed by conducting pre-hybridization at 68° C. for 30 min or longer using “Rapid-hyb buffer” (Amersham LIFE SCIENCE), adding a labeled probe, and warming at 68° C. for 1 hour or longer. The following washing step can be conducted, for example, in a low stringent condition. An exemplary low stringent condition may include 42° C., 2×SSC, 0.1% SDS, preferably 50° C., 2×SSC, 0.1% SDS. High stringency conditions are often preferably used. An exemplary high stringency condition may include washing 3 times in 2×SSC, 0.01% SDS at room temperature for 20 min, then washing 3 times in 1×SSC, 0.1% SDS at 37° C. for 20 min, and washing twice in 1×SSC, 0.1% SDS at 50° C. for 20 min. However, several factors, such as temperature and salt concentration, can influence the stringency of hybridization and one skilled in the art can suitably select the factors to achieve the requisite stringency.

Generally, it is known that modifications of one or more amino acid in a protein do not influence the function of the protein. In fact, mutated or modified proteins, proteins having amino acid sequences modified by substituting, deleting, inserting, and/or adding one or more amino acid residues of a certain amino acid sequence, have been known to retain the original biological activity (Mark et al., Proc Natl Acad Sci USA 81: 5662-6 (1984); Zoller and Smith, Nucleic Acids Res 10:6487-500 (1982); Dalbadie-McFarland et al., Proc Natl Acad Sci USA 79: 6409-13 (1982)). Accordingly, one of skill in the art will recognize that individual additions, deletions, insertions, or substitutions to an amino acid sequence which alter a single amino acid or a small percentage of amino acids or those considered to be a “conservative modifications”, wherein the alteration of a protein results in a protein with similar functions, are acceptable in the context of the instant invention.

So long as the activity the protein is maintained, the number of amino acid mutations is not particularly limited. However, it is generally preferred to alter 5% or less of the amino acid sequence. Accordingly, in a preferred embodiment, the number of amino acids to be mutated in such a mutant is generally 30 amino acids or less, preferably 20 amino acids or less, more preferably 10 amino acids or less, more preferably 6 amino acids or less, and even more preferably 3 amino acids or less.

An amino acid residue to be mutated is preferably mutated into a different amino acid in which the properties of the amino acid side-chain are conserved (a process known as conservative amino acid substitution). Examples of properties of amino acid side chains are hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W). Conservative substitution tables providing functionally similar amino acids are well known in the art. For example, the following eight groups each contain amino acids that are conservative substitutions for one another:

### 1) Alanine (A), Glycine (G);

2) Aspartic acid (D), Glutamic acid (E);

### 3) Aspargine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

8) Cysteine (C), Methionine (M) (see, e.g., Creighton, Proteins 1984).

Such conservatively modified polypeptides are included in the present ECT2 protein. However, the present invention is not restricted thereto and the ECT2 protein includes non-conservative modifications, so long as at least one biological activity of the ECT2 protein is retained. Furthermore, the modified proteins do not exclude polymorphic variants, interspecies homologues, and those encoded by alleles of these proteins.

Moreover, the ECT2 gene of the present invention encompasses polynucleotides that encode such functional equivalents of the ECT2 protein. In addition to hybridization, a gene amplification method, for example, the polymerase chain reaction (PCR) method, can be utilized to isolate a polynucleotide encoding a polypeptide functionally equivalent to the ECT2 protein, using a primer synthesized based on the sequence information of the protein encoding DNA (SEQ ID NO: 11). Polynucleotides and polypeptides that are functionally equivalent to the human ECT2 gene and protein, re-spectively, normally have a high homology to the originating nucleotide or amino acid sequence thereof. “High homology” typically refers to a homology of 40% or higher, preferably 60% or higher, more preferably 80% or higher, even more preferably 90% to 95% or higher. The homology of a particular polynucleotide or polypeptide can be determined by following the algorithm in “Wilbur and Lipman, Proc Natl Acad Sci USA 80: 726-30 (1983)”.

I. Double-Stranded Molecule:

As use herein, the term “double-stranded molecule” refers to a nucleic acid molecule that inhibits expression of a target gene including, for example, short interfering RNA (siRNA; e.g., double-stranded ribonucleic acid (dsRNA) or small hairpin RNA (shRNA)) and short interfering DNA/RNA (siD/R-NA; e.g. double-stranded chimera of DNA and RNA (dsD/R-NA) or small hairpin chimera of DNA and RNA (shD/R-NA)).

As used herein, the term “dsRNA” refers to a construct of two RNA molecules comprising complementary sequences to one another and that have annealed together via the complementary sequences to form a double-stranded RNA molecule. The nu-cleotide sequence of two strands may comprise not only the “sense” or “antisense” RNAs selected from a protein coding sequence of target gene sequence, but also RNA molecule having a nucleotide sequence selected from non-coding region of the target gene.

The term “shRNA”, as used herein, refers to an siRNA having a stem-loop structure, comprising a first and second regions complementary to one another, i.e., sense and antisense strands. The degree of complementarity and orientation of the regions are sufficient such that base pairing occurs between the regions, the first and second regions being joined by a loop region, the loop resulting from a lack of base pairing between nucleotides (or nucleotide analogs) within the loop region. The loop region of an shRNA is a single-stranded region intervening between the sense and antisense strands and may also be referred to as “intervening single-strand”.

As used herein, the term “siD/R-NA” refers to a double-stranded polynucleotide molecule which is composed of both RNA and DNA, and includes hybrids and chimeras of RNA and DNA and prevents translation of a target mRNA. Herein, a hybrid indicates a molecule wherein a polynucleotide composed of DNA and a polynu-cleotide composed of RNA hybridize to each other to form the double-stranded molecule; whereas a chimera indicates that one or both of the strands composing the double stranded molecule may contain RNA and DNA. Standard techniques of introducing siD/R-NA into the cell are used. The siD/R-NA includes a sense nucleic acid sequence (also referred to as “sense strand”), an antisense nucleic acid sequence (also referred to as “antisense strand”) or both. The siD/R-NA may be constructed such that a single transcript has both the sense and complementary antisense nucleic acid sequences from the target gene, e.g., a hairpin. The siD/R-NA may either be a dsD/R-NA or shD/R-NA.

As used herein, the term “dsD/R-NA” refers to a construct of two molecules comprising complementary sequences to one another and that have annealed together via the complementary sequences to form a double-stranded polynucleotide molecule. The nucleotide sequence of two strands may comprise not only the “sense” or “antisense” polynucleotides sequence selected from a protein coding sequence of target gene sequence, but also polynucleotide having a nucleotide sequence selected from non-coding region of the target gene. One or both of the two molecules constructing the dsD/R-NA are composed of both RNA and DNA (chimeric molecule), or alternatively, one of the molecules is composed of RNA and the other is composed of DNA (hybrid double-strand).

The term “shD/R-NA”, as used herein, refers to an siD/R-NA having a stem-loop structure, comprising a first and second regions complementary to one another, i.e., sense and antisense strands. The degree of complementarity and orientation of the regions are sufficient such that base pairing occurs between the regions, the first and second regions being joined by a loop region, the loop resulting from a lack of base pairing between nucleotides (or nucleotide analogs) within the loop region. The loop region of an shD/R-NA is a single-stranded region intervening between the sense and antisense strands and may also be referred to as “intervening single-strand”.

The double-stranded molecules of the invention may contain one or more modified nucleotides and/or non-phosphodiester linkages. Chemical modifications well known in the art are capable of increasing stability, availability, and/or cell uptake of the double-stranded molecule. The skilled person will be aware of other types of chemical modification which may be incorporated into the present molecules (e.g., WO03/070744; WO2005/045037). In one embodiment, modifications can be used to provide improved resistance to degradation or improved uptake. Examples of such modifications include phosphorothioate linkages, 2′-O-methyl ribonucleotides (especially on the sense strand of a double-stranded molecule), 2′-deoxy-fluoro ribonucleotides, 2′-deoxy ribonucleotides, “universal base” nucleotides, 5′-C-methyl nucleotides, and inverted deoxyabasic residue incorporation (US20060122137).

In another embodiment, modifications can be used to enhance the stability or to increase targeting efficiency of the double-stranded molecule. Modifications include chemical cross linking between the two complementary strands of a double-stranded molecule, chemical modification of a 3′ or 5′ terminus of a strand of a double-stranded molecule, sugar modifications, nucleobase modifications and/or backbone modifications, 2-fluoro modified ribonucleotides and 2′-deoxy ribonucleotides (WO2004/029212). In another embodiment, modifications can be used to increased or decreased affinity for the complementary nucleotides in the target mRNA and/or in the complementary double-stranded molecule strand (WO2005/044976). For example, an unmodified pyrimidine nucleotide can be substituted for a 2-thio, 5-alkynyl, 5-methyl, or 5-propynyl pyrimidine. Additionally, an unmodified purine can be substituted with a 7-deaza, 7-alkyl, or 7-alkenyl purine. In another embodiment, when the double-stranded molecule is a double-stranded molecule with a 3′ overhang, the 3′-terminal nucleotide overhanging nucleotides may be replaced by deoxyribonucleotides (Elbashir S M et al., Genes Dev 2001 Jan. 15, 15(2): 188-200). For further details, published documents such as US20060234970 are available. The present invention is not limited to these examples and any known chemical modifications may be employed for the double-stranded molecules of the present invention so long as the resulting molecule retains the ability to inhibit the expression of the target gene.

Furthermore, the double-stranded molecules of the present invention may comprise both DNA and RNA, e.g., dsD/R-NA or shD/R-NA. Specifically, a hybrid polynucleotide of a DNA strand and an RNA strand or a DNA-RNA chimera polynucleotide shows increased stability. Mixing of DNA and RNA, i.e., a hybrid type double-stranded molecule consisting of a DNA strand (polynucleotide) and an RNA strand (polynucleotide), a chimera type double-stranded molecule comprising both DNA and RNA on any or both of the single strands (polynucleotides), or the like may be formed for enhancing stability of the double-stranded molecule. The hybrid of a DNA strand and an RNA strand may be the hybrid in which either the sense strand is DNA and the antisense strand is RNA, or the opposite so long as it has an activity to inhibit expression of the target gene when introduced into a cell expressing the gene. Preferably, the sense strand polynucleotide is DNA and the antisense strand polynucleotide is RNA. Also, the chimera type double-stranded molecule may be either where both of the sense and antisense strands are composed of DNA and RNA, or where any one of the sense and antisense strands is composed of DNA and RNA so long as it has an activity to inhibit expression of the target gene when introduced into a cell expressing the gene.

In order to enhance stability of the double-stranded molecule, the molecule preferably contains as much DNA as possible, whereas to induce inhibition of the target gene expression, the molecule is required to be RNA within a range to induce sufficient inhibition of the expression. As a preferred example of the chimera type double-stranded molecule, an upstream partial region (i.e., a region flanking to the target sequence or complementary sequence thereof within the sense or antisense strands) of the double-stranded molecule is RNA. Preferably, the upstream partial region indicates the 5′ side (5′-end) of the sense strand and the 3′ side (3′-end) of the antisense strand. That is, in preferable embodiments, a region flanking to the 3′-end of the antisense strand, or both of a region flanking to the 5′-end of sense strand and a region flanking to the 3′-end of antisense strand consists of RNA. For instance, the chimera or hybrid type double-stranded molecule of the present invention comprise following combinations.

sense strand: 5′-[DNA]-3′

3′-(RNA)-[DNA]-5′: antisense strand,

sense strand: 5′-(RNA)-[DNA]-3′

3′-(RNA)-[DNA]-5′: antisense strand, and

sense strand: 5′-(RNA)-[DNA]-3′

3′-(RNA)-5′: antisense strand.

The upstream partial region preferably is a domain consisting of 9 to 13 nucleotides counted from the terminus of the target sequence or complementary sequence thereto within the sense or antisense strands of the double-stranded molecules. Moreover, preferred examples of such chimera type double-stranded molecules include those having a strand length of 19 to 21 nucleotides in which at least the upstream half region (5′ side region for the sense strand and 3′ side region for the antisense strand) of the polynucleotide is RNA and the other half is DNA. In such a chimera type double-stranded molecule, the effect to inhibit expression of the target gene is much higher when the entire antisense strand is RNA (US20050004064).

In the present invention, the double-stranded molecule may form a hairpin, such as a short hairpin RNA (shRNA) and short hairpin consisting of DNA and RNA (shD/R-NA). The shRNA or shD/R-NA is a sequence of RNA or mixture of RNA and DNA making a tight hairpin turn that can be used to silence gene expression via RNA interference. The shRNA or shD/R-NA comprises the sense target sequence and the antisense target sequence on a single strand wherein the sequences are separated by a loop sequence. Generally, the hairpin structure is cleaved by the cellular machinery into dsRNA or dsD/R-NA, which is then bound to the RNA-induced silencing complex (RISC). This complex binds to and cleaves mRNAs which match the target sequence of the dsRNA or dsD/R-NA.

A double-stranded molecule against the ECT2 gene (e.g. ‘ECT2 siRNA’) can be used to reduce the expression level of the gene. Herein, the term “siRNA” refers to a double-stranded RNA molecule which prevents translation of a target mRNA. In the context of the present invention, the double-stranded molecule is composed of a sense nucleic acid sequence and an anti-sense nucleic acid sequence against the up-regulated marker gene, ECT2. The double-stranded molecule is constructed so that it includes both a sense and complementary antisense sequences of the target gene, i.e., a nucleotide having a hairpin structure. The double-stranded molecule may either be a dsRNA, shRNA, dsD/RNA or shD/RNA.

A double-stranded molecule of the ECT2 gene hybridizes to target mRNA, i.e., associates with the normally single-stranded mRNA transcript and thereby interfering with translation of the mRNA, which finally decreases or inhibits production (expression) of the polypeptide encoded by the gene. Thus, an siRNA molecule of the invention can be defined by its ability to specifically hybridize to the mRNA of the ECT2 gene under stringent conditions.

In the context of the present invention, a double-stranded molecule is preferably less than 500, 200, 100, 50, or 25 nucleotides in length. More preferably a double-stranded molecule is 19-25 nucleotides in length. Exemplary target nucleic acid sequences of ECT2 double-stranded molecule include the oligonucleotide sequences corresponding to SEQ ID NO: 1 or 2. Therefore, preferable double-stranded molecule of the present invention comprises a sense strand and an antisense strand complementary thereto, hybridized to each other to form the double-stranded molecule and targets to a nucleotide sequence selected from the group consisting of SEQ ID NOs: 1 and 2, and wherein the double-stranded molecule, when introduced into a cell expressing the ETC2 gene, inhibits expression of the gene. The sense strand comprises a nucleotide sequence corresponding to a target sequence. Preferably, the target sequence comprises from about 19 to about 25 contiguous nucleotides from the nucleotide sequences of SEQ ID NO: 11. More preferably, the target sequence consists of from about 19 to about 25 contiguous nucleotides from the nucleotide sequences of SEQ ID NO: 11. The double-stranded molecule may be a single oligonucleotide molecule comprising the sense strand and the antisense strand linked via a single-stranded oligonucleotide sequence.

The nucleotide “t” in the sequence should be replaced with “u” in RNA or derivatives thereof. Accordingly, for example, the present invention provides double-stranded RNA molecules having the oligonucleotide sequence

| | |
|---|---|
| 5′-gauaaaggaugaucuugaa-3′ | (SEQ ID NO: 1) |
| or | |
| 5′-cagaggagauuaagacuau-3′. | (SEQ ID NO: 2) |

In order to enhance the inhibition activity of the double-stranded molecules, nucleotide “u” can be added to the 3′ end of the antisense strand. The number of “u”s to be added is at least 2, generally 2 to 10, preferably 2 to 5. The added “u”s form a single strand at the 3′ end of the antisense strand of the double-stranded molecule.

A loop sequence composed of an arbitrary nucleotide sequence can be located between the sense and antisense sequence in order to form the hairpin loop structure. Thus, the present invention also provides double-stranded molecule having the general formula 5′-[A]-[B]-[A′]-3′, wherein [A] is a oligonucleotide sequence corresponding to a sequence that specifically hybridizes to an mRNA or a cDNA of the ECT2 gene. In preferred embodiments, [A] is a nucleotide sequence corresponding to a sequence of the ECT2 gene (e.g., SEQ ID NO: 1); [B] is a nucleotide sequence composed of 3 to 23 nucleotides; and [A′] is a nucleotide sequence composed of the complementary sequence of [A]. The region [A] hybridizes to [A′], and then a loop composed of region [B] is formed. The loop sequence may be preferably 3 to 23 nucleotide in length. The loop sequence, for example, can be selected from a group composed of following sequences (see, Ambion website on the worldwide web at ambion.com/techlib/tb/tb_{—}506.html):

CCC, CCACC, or CCACACC: Jacque J M et al., Nature 2002, 418: 435-8.

UUCG: Lee N S et al., Nature Biotechnology 2002, 20:500-5; Fruscoloni P et al., Proc Natl Acad Sci USA 2003, 100(4):1639-44.

UUCAAGAGA: Dykxhoorn D M et al., Nature Reviews Molecular Cell Biology 2003, 4:457-67.

‘UUCAAGAGA (“ttcaagaga” in DNA)’ is a particularly suitable loop sequence. Furthermore, loop sequence consisting of 23 nucleotides also provides an active siRNA (Jacque J-M et al., Nature 2002, 418:435-8).

Exemplary hairpin double-stranded molecule suitable for use in the context of the present invention include,

(for target sequence of SEQ ID NO: 1)
5′-gauaaaggaugaucuugaa-[b]-uucaagaucauccuuuauc-3′;
and
(for target sequence of SEQ ID NO: 2)
5′-cagaggagauuaagacua-[b]-uagucuuaaucuccucug-3′.

The oligonucleotide sequence of suitable double-stranded molecules can be designed using an siRNA design computer program available from the Ambion website (ambion.com/techlib/misc/siRNA_finder.html). The computer program selects nucleotide sequences for double-stranded molecule synthesis based on the following protocol.

Selection of siRNA Target Sites:

1. Beginning with the AUG start codon of the object transcript, scan downstream for AA dinucleotide sequences. Record the occurrence of each AA and the 3′ adjacent 19 nucleotides as potential target sites. Tuschl et al. Genes Cev 1999, 13(24):3191-7 do not recommend designing target sequence to the 5′ and 3′ untranslated regions (UTRs) and regions near the start codon (within 75 nucleotides) as these may be richer in regulatory protein binding sites. UTR-binding proteins and/or translation initiation complexes may interfere with binding of the endonuclease complex.

2. Compare the potential target sites to the human genome database and eliminate from consideration any target sequences with significant homology to other coding sequences. The homology search can be performed using BLAST (Altschul S F et al., Nucleic Acids Res 1997, 25:3389-402; J Mol Biol 1990, 215:403-10.), which can be found on the NCBI server on the worldwide web at: ncbi.nlm.nih.gov/BLAST/.

3. Select qualifying target sequences for synthesis. At Ambion, preferably several target sequences can be selected along the length of the gene to evaluate.

Standard techniques for introducing a double-stranded molecule into the cell may be used. For example, a double-stranded molecule of ECT2 can be directly introduced into the cells in a form that is capable of binding to the mRNA transcripts. In these embodiments, the double-stranded molecules of the present invention are typically modified as described above for antisense molecules. Other modifications are also possible, for example, cholesterol-conjugated double-stranded molecules have shown improved pharmacological properties (Song et al., Nature Med 2003, 9:347-51).

Alternatively, a DNA encoding the double-stranded molecule may be carried in a vector (hereinafter, also referred to as ‘siRNA vector’). Such vectors may be produced, for example, by cloning the target ECT2 gene sequence into an expression vector having operatively-linked regulatory sequences (e.g., a RNA polymerase III transcription unit from the small nuclear RNA (snRNA) U6 or the human H1 RNA promoter) flanking the sequence in a manner that allows for expression (by transcription of the DNA molecule) of both strands (Lee N S et al., Nature Biotechnology 2002, 20: 500-5). For example, an RNA molecule that is antisense to mRNA of the ECT2 gene is transcribed by a first promoter (e.g., a promoter sequence 3′ of the cloned DNA) and an RNA molecule that is the sense strand for the mRNA of the ECT2 gene is transcribed by a second promoter (e.g., a promoter sequence 5′ of the cloned DNA). The sense and antisense strands hybridize in vivo to generate double-stranded molecule constructs for silencing the expression of the ECT2 gene. Alternatively, the two constructs can be utilized to create the sense and anti-sense strands of a single-stranded construct. In this case, a construct having secondary structure, e.g., hairpin, is produced as a single transcript that includes both the sense and complementary antisense sequences of the target gene.

For introducing the vector of double-stranded molecule into the cell, transfection-enhancing agent can be used. FuGENE6 (Roche diagnostics), Lipofectamine 2000 (Invitrogen), Oligofectamine (Invitrogen), and Nucleofector (Wako pure Chemical) are useful as the transfection-enhancing agent. Therefore, the present pharmaceutical composition may further include such transfection-enhancing agents.

II. Antibody:

The present invention provides antibodies against an ECT2 protein or fragments of the antibodies. In other words, the antibodies of the present invention can be used for detecting an ECT2 specific expression. Therefore, the antibodies of the present invention are useful for diagnosing ECT2 related diseases, for example lung and esophageal cancer and treating those diseases. The antibody can be prepared by using ECT2 protein or fragments thereof (e.g. COOH-terminal portion of ECT2 corresponding to codons 703-883 (SEQ ID NO: 8)) (see the item of ‘D. Preparation of anti-ECT2 polyclonal antibody’ in EXAMPLE). Therefore, the preferred embodiment of the present invention is an antibody recognizing ECT2 which binds the antigen comprising a peptide having an amino acid sequence of SEQ ID NO: 8.

When the expression of ECT2 is observed by tissue immunostaining, the survival rate is low in the patient with lung and esophageal cancer, as shown in Table 1 and 2. This finding suggests that the expression of ECT2 should be useful in diagnosing malignant prognosis as an index. Therefore, prognosis may be diagnosed more accurately using the ECT2 specific antibody.

Furthermore, the antibody of the present invention must be an useful tool for functional analysis of ECT2. The term “antibody” as used herein encompasses naturally occurring antibodies as well as non-naturally occurring antibodies, including, for example, single chain antibodies, chimeric, bifunctional and humanized antibodies, as well as antigen-binding fragments thereof, (e.g., Fab′, F(ab′)₂, Fab, Fv and rIgG). See also, Pierce Catalog and Handbook, 1994-1995 (Pierce Chemical Co., Rockford, Ill.). See also, e.g. Kuby, J., Immunology, 3rd Ed., W.H. Freeman & Co., New York (1998). Such non-naturally occurring antibodies can be constructed using solid phase peptide synthesis, can be produced recombinantly or can be obtained, for example, by screening combinatorial libraries consisting of variable heavy chains and variable light chains as described by Huse et al., Science 246:1275-81 (1989), which is incorporated herein by reference. These and other methods of making, for example, chimeric, humanized, CDR-grafted, single chain, and bifunctional antibodies are well known to those skilled in the art (Winter and Harris, Immunol. Today 14:243-6 (1993); Ward et al., Nature 341:544-6 (1989); Harlow and Lane, Antibodies, 511-52, Cold Spring Harbor Laboratory publications, New York, 1988; Hilyard et al., Protein Engineering: A practical approach (IRL Press 1992); Borrebaeck, Antibody Engineering, 2d ed. (Oxford University Press 1995); each of which is incorporated herein by reference).

The term “antibody” includes both polyclonal and monoclonal antibodies. The term also includes genetically engineered forms such as chimeric antibodies (e.g., humanized murine antibodies) and heteroconjugate antibodies (e.g., bispecific antibodies). The term also refers to recombinant single chain Fv fragments (scFv). The term antibody also includes bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Holliger et al. (1993) Proc Natl Acad Sci USA. 90:6444, Gruber et al. (1994) J Immunol:5368, Zhu et al. (1997) Protein Sci 6:781, Hu et al. (1997) Cancer Res. 56:3055, Adams et al. (1993) Cancer Res. 53:4026, and McCartney, et al. (1995) Protein Eng. 8:301.

Typically, an antibody has a heavy and light chain. Each heavy and light chain contains a constant region and a variable region, (the regions are also known as “domains”). Light and heavy chain variable regions contain four “framework” regions interrupted by three hyper-variable regions, also called “complementarity-determining regions” or “CDRs”. The extent of the framework regions and CDRs have been defined. The sequences of the framework regions of different light or heavy chains are relatively conserved within a species. The framework region of an antibody, that is the combined framework regions of the constituent light and heavy chains, serves to position and align the CDRs in three dimensional spaces.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found.

References to “VH” refer to the variable region of an immunoglobulin heavy chain of an antibody, including the heavy chain of an Fv, scFv, or Fab. References to “VL” refer to the variable region of an immunoglobulin light chain, including the light chain of an Fv, scFv, dsFv or Fab.

The phrase “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site.

A “chimeric antibody” is an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

A “humanized antibody” is an immunoglobulin molecule that contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature 321:522-5 (1986); Riechmann et al., Nature 332:323-7 (1988); and Presta, Curr. Op. Struct. Biol. 2:593-6 (1992)). Humanization can be essentially performed following the method of Winter and co-workers (Jones et al., Nature 321:522-5 (1986); Riechmann et al., Nature 332:323-7 (1988); Verhoeyen et al., Science 239:1534-6 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

The terms “epitope”, “antigenic” and “determinant” refer to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, X-ray crystallography and 2-dimensional nuclear magnetic resonance. See, e.g., Epitope Mapping Protocols in Methods in Molecular Biology, Vol. 66, Glenn E. Morris, Ed (1996).

The terms “non-antibody binding protein” or “non-antibody ligand” or “antigen binding protein” interchangeably refer to antibody mimics that use non-immunoglobulin protein scaffolds, including adnectins, avimers, single chain polypeptide binding molecules, and antibody-like binding peptidomimetics, as discussed in more detail below.

Other compounds have been developed that target and bind to targets in a manner similar to antibodies. Certain of these “antibody mimics” use non-immunoglobulin protein scaffolds as alternative protein frameworks for the variable regions of antibodies.

For example, Ladner et al. (U.S. Pat. No. 5,260,203) describe single polypeptide chain binding molecules with binding specificity similar to that of the aggregated, but molecularly separate, light and heavy chain variable region of antibodies. The single-chain binding molecule contains the antigen binding sites of both the heavy and light variable regions of an antibody connected by a peptide linker and will fold into a structure similar to that of the two peptide antibody. The single-chain binding molecule displays several advantages over conventional antibodies, including, smaller size, greater stability and are more easily modified.

Ku et al. (Proc. Natl. Acad. Sci. USA 92(14):6552-6556 (1995)) discloses an alternative to antibodies based on cytochrome b562. Ku et al. (1995) generated a library in which two of the loops of cytochrome b562 were randomized and selected for binding against bovine serum albumin. The individual mutants were found to bind selectively with BSA similarly with anti-BSA antibodies.

Lipovsek et al. (U.S. Pat. Nos. 6,818,418 and 7,115,396) discloses an antibody mimic featuring a fibronectin or fibronectin-like protein scaffold and at least one variable loop. Known as Adnectins, these fibronectin-based antibody mimics exhibit many of the same characteristics of natural or engineered antibodies, including high affinity and specificity for any targeted ligand. Any technique for evolving new or improved binding proteins can be used with these antibody mimics.

The structure of these fibronectin-based antibody mimics is similar to the structure of the variable region of the IgG heavy chain. Therefore, these mimics display antigen binding properties similar in nature and affinity to those of native antibodies. Further, these fibronectin-based antibody mimics exhibit certain benefits over antibodies and antibody fragments. For example, these antibody mimics do not rely on disulfide bonds for native fold stability, and are, therefore, stable under conditions which would normally break down antibodies. In addition, since the structure of these fibronectin-based antibody mimics is similar to that of the IgG heavy chain, the process for loop randomization and shuffling can be employed in vitro that is similar to the process of affinity maturation of antibodies in vivo.

Beste et al. (Proc. Natl. Acad. Sci. USA 96(5):1898-1903 (1999)) discloses an antibody mimic based on a lipocalin scaffold (Anticalin (registered trademark)). Lipocalins are composed of a beta-barrel with four hypervariable loops at the terminus of the protein. Beste (1999), subjected the loops to random mutagenesis and selected for binding with, for example, fluorescein. Three variants exhibited specific binding with fluorescein, with one variant showing binding similar to that of an anti-fluorescein antibody. Further analysis revealed that all of the randomized positions are variable, indicating that Anticalin (registered trademark) would be suitable to be used as an alternative to antibodies.

Anticalins (registered trademark) are small, single chain peptides, typically between 160 and 180 residues, which provides several advantages over antibodies, including decreased cost of production, increased stability in storage and decreased immunological reaction.

Hamilton et al. (U.S. Pat. No. 5,770,380) discloses a synthetic antibody mimic using the rigid, non-peptide organic scaffold of calixarene, attached with multiple variable peptide loops used as binding sites. The peptide loops all project from the same side geometrically from the calixarene, with respect to each other. Because of this geometric confirmation, all of the loops are available for binding, increasing the binding affinity to a ligand. However, in comparison to other antibody mimics, the calixarene-based antibody mimic does not consist exclusively of a peptide, and therefore it is less vulnerable to attack by protease enzymes. Neither does the scaffold consist purely of a peptide, DNA or RNA, meaning this antibody mimic is relatively stable in extreme environmental conditions and has a long life span. Further, since the calixarene-based antibody mimic is relatively small, it is less likely to produce an immunogenic response.

Murali et al. (Cell. Mol. Biol. 49(2):209-216 (2003)) discusses a methodology for reducing antibodies into smaller peptidomimetics, they term “antibody like binding peptidomemetics” (ABiP) which can also be useful as an alternative to antibodies.

Silverman et al. (Nat. Biotechnol. (2005), 23: 1556-1561) discloses fusion proteins that are single-chain polypeptides comprising multiple domains termed “avimers”. Developed from human extracellular receptor domains by in vitro exon shuffling and phage display the avimers are a class of binding proteins somewhat similar to antibodies in their affinities and specificities for various target molecules. The resulting multidomain proteins can comprise multiple independent binding domains that can exhibit improved affinity (in some cases sub-nanomolar) and specificity compared with single-epitope binding proteins. Additional details concerning methods of construction and use of avimers are disclosed, for example, in U.S. Patent App. Pub. Nos. 20040175756, 20050048512, 20050053973, 20050089932 and 20050221384.

In addition to non-immunoglobulin protein frameworks, antibody properties have also been mimicked in compounds comprising RNA molecules and unnatural oligomers (e.g., protease inhibitors, benzodiazepines, purine derivatives and beta-turn mimics) all of which are suitable for use with the present invention.

III. Diagnosing Lung Cancer and Esophageal Cancer

The expression of the ECT2 gene was found to be specifically elevated in patients with lung cancer or esophageal cancer. Therefore, the gene identified herein, as well as its transcription and translation products, find diagnostic utility as a marker for cancer. More particularly, by measuring the expression of the ECT2 gene in a cell sample, lung cancer or esophageal cancer can be diagnosed. Thus, the present invention provides a method for diagnosing lung cancer or esophageal cancer or a predisposition for developing lung cancer or esophageal cancer in a subject by determining the expression level of the ECT2 gene in the subject.

According to the present invention, an intermediate result for examining the condition of a subject may be provided. Such intermediate result may be combined with additional information to assist a doctor, nurse, or other practitioner to determine that a subject suffers from lung cancer or esophageal cancer. That is, the present invention provides a diagnostic marker ECT2 for examining cancer. Alternatively, the present invention may be used to detect cancerous cells in a subject-derived tissue, and provide a doctor with useful information to determine that the subject suffers from lung cancer or esophageal cancer.

The diagnostic method of the present invention involves the step of determining (e.g., measuring) the expression of an ECT2 gene. Using sequence information provided by the GenBank™ database entries for known sequences, the ECT2 gene can be detected and measured using conventional techniques well known to one of ordinary skill in the art. For example, sequences within the sequence database entries corresponding to the ECT2 gene can be used to construct probes for detecting RNA sequences corresponding to the ECT2 gene in, e.g., Northern blot hybridization analyses. Hybridization probes typically include at least 10, at least 20, at least 50, at least 100, or at least 200 consecutive nucleotides of an ECT2 sequence. As another example, the sequences can be used to construct primers for specifically amplifying the ECT2 nucleic acid in, e.g., amplification-based detection methods, for example, reverse-transcription based polymerase chain reaction. As another example, an antibody against ECT2, e.g., an anti-ECT2 polyclonal antibody or anti-ECT2 monoclonal antibody, can be used for immunoassay, for example, immunohisto-chemical analysis, western blot analysis or ELISA, etc.

The level of the ECT2 gene expression detected in a test cell population, e.g., a tissue sample from a patient, can then be compared to the expression level(s) of the gene in a reference cell population. The reference cell population may include one or more cells for which the compared parameter is known, i.e., non-small lung cancer cells (e.g, LC cells), esophageal squamous-cell carcinoma cells (e.g., EC cells), normal lung epithelial cells (e.g., non-LC cells) or normal esophageal epithelial cells (e.g., non-EC cells).

Whether or not a level of gene expression in a test cell population as compared to a reference cell population indicates the presence of LC, EC or a predisposition thereto depends upon the composition of the reference cell population. For example, if the reference cell population is composed of non-LC cells or non-EC cells, a similarity in gene expression level between the test cell population and the reference cell population indicates the test cell population is non-LC or non-EC. Conversely, if the reference cell population is made up of LC cells or EC cells, a similarity in gene expression between the test cell population and the reference cell population indicates that the test cell population includes LC cells or EC cells.

A level of expression of an ECT2 gene in a test cell population is considered “altered” or deemed to “differ” if it varies from the expression level of the ECT2 gene in a reference cell population by more than 1.1, more than 1.5, more than 2.0, more than 5.0, more than 10.0 or more fold.

Differential gene expression between a test cell population and a reference cell population can be normalized to a control nucleic acid, e.g. a housekeeping gene. For example, a control nucleic acid is one which is known not to differ depending on the cancerous or non-cancerous state of the cell. The expression level of a control nucleic acid can thus be used to normalize signal levels in the test and reference cell populations. Exemplary control genes include, but are not limited to, e.g., beta actin, glyceraldehyde 3-phosphate dehydrogenase and ribosomal protein P1.

The test cell population can be compared to multiple reference cell populations. Each of the multiple reference cell populations can differ in the known parameter. Thus, a test cell population can be compared to a first reference cell population known to contain, e.g., LC cells or EC cells, as well as a second reference cell population known to contain, e.g., non-LC cells or non-EC cells (normal cells). The test cell population can be included in a tissue or cell sample from a subject known to contain, or suspected of containing, LC cells or EC cells.

The test cell population can be obtained from a bodily tissue or a bodily fluid, e.g., biological fluid (for example, blood, sputum, saliva). For example, the test cell population can be purified from lung tissue or esophageal tissue. Preferably, the test cell population comprises an epithelial cell. The epithelial cell is preferably from a tissue known to be or suspected to be a non-small cell carcinoma or an esophageal squamous-cell carcinoma.

Cells in the reference cell population are preferably from a tissue type similar to that of the test cell population. Optionally, the reference cell population is a cell line, e.g. an LC cell line or an EC cell line (i.e., a positive control) or a normal non-LC cell line or a non-EC cell line (i.e., a negative control). Alternatively, the control cell population can be from a database of molecular information obtained from cells for which the assayed parameter or condition is known.

The subject is preferably a mammal. Exemplary mammals include, but are not limited to, e.g., a human, non-human primate, mouse, rat, dog, cat, horse, or cow.

Expression of the ECT2 gene disclosed herein can be determined at the protein or nucleic acid level, using methods known in the art. For example, Northern hybridization analysis, using probes which specifically recognize one or more of these nucleic acid sequences, can be used to determine gene expression. Alternatively, gene expression can be measured using reverse-transcription-based PCR assays, using primers specific for the ECT2 gene sequence e.g., SEQ ID NO: 3 and 4. Expression can also be determined at the protein level, i.e., by measuring the level of a polypeptide encoded by an ECT2 gene, or the biological activity thereof. Such methods are well known in the art and include, but are not limited to, e.g., immunoassays that utilize antibodies to proteins encoded by the genes, e.g., anti-ECT2 polyclonal antibodies which recognized amino acid sequence comprising SEQ ID NO: 8 or 12 described in Example 1, but not limited. The biological activities of the proteins encoded by the genes are generally well known and include, e.g., cell proliferative activity. See, Sambrook and Russell, Molecular Cloning: A Laboratory Manual, 3rd Edition, 2001, Cold Spring Harbor Laboratory Press; Ausubel, Current Protocols in Molecular Biology, 1987-2006, John Wiley and Sons; and Harlow and Lane, Using Antibodies: A Laboratory Manual, 1998, Cold Spring Harbor Laboratory Press.

In the context of the present invention, EC or LC may be diagnosed by measuring the expression level of ECT2 nucleic acids in a test population of cells, (i.e., a biological sample from a patient). Preferably, the test cell population contains an epithelial cell, e.g., a cell obtained from lung tissue or esophageal tissue. Gene expression can also be measured from blood or other bodily fluids, for example, saliva or sputum. Other biological samples can be used for measuring protein levels. For example, the protein level in blood or serum from a subject to be diagnosed can be measured by immunoassay or other conventional biological assay.

Expression of the ECT2 gene is first determined in the test cell population or biological sample and then compared to the normal control expression level of the ECT2 gene. A normal control level corresponds to an expression of the ECT2 gene typically found in a cell population from a subject known not to be suffering from LC or EC. An alteration or difference (e.g., an increase) in the level of expression of the ECT2 gene in a tissue sample from a patient in comparison to expression from a normal control sample indicates that the subject is suffering from or is at risk of developing LC or EC. For example, an increase in the expression of the ECT2 gene in the test cell population as compared to the expression in a normal control cell population indicates that the subject is suffering from or is at risk of developing LC or EC.

An increase in expression levels of the ECT2 gene in the test cell population as compared to normal control expression levels indicates that the subject suffers from or is at risk of developing LC or EC. For example, increase in expression levels of at least 1%, at least 5%, at least 25%, at least 50%, at least 60%, at least 70%, at least 80%, at least 90% or more of the level of the ECT2 gene indicates that the subject suffers from or is at risk of developing LC or EC.

IV. Screening Assays Identifying Agents that Inhibit ECT2 Gene Expression:

An agent that inhibits the expression of the ECT2 gene or the activity of its gene product can be identified by contacting a test cell population that expresses the ECT2 gene with a test agent and then determining the subsequent level of gene expression or activity of its gene product. A decrease in the level of gene expression or of activity of its gene product in the presence of the agent as compared to the expression or activity level in the absence of the test agent indicates that the agent is an inhibitor of the ECT2 gene and therefore useful in inhibiting LC and EC.

The test cell population can include any cells expressing the ECT2 gene. For example, the test cell population can contain epithelial cells, for example, cells from lung tissue or esophageal tissue. Furthermore, the test cell population can be an immortalized cell line from a non-small lung cancer cell or an esophageal squamous-cell carcinoma cell. Alternatively, the test cell population can be composed of cells which have been transfected with the ECT2 gene or which have been transfected with a regulatory sequence (e.g., promoter sequence) from the ECT2 gene operably linked to a reporter gene.

The agent can be, for example, an inhibitory oligonucleotide (e.g., an antisense oligonucleotide, an siRNA or a ribozyme), an antibody, a polypeptide or a small organic molecule. Screening for suitable inhibitory agents can be carried out using high throughput methods, by simultaneously screening a plurality of agents using multiwell plates (e.g., 96-well, 192-well, 384-well, 768-well, 1536-well). Automated systems for high throughput screening are commercially available from, for example, Caliper Life Sciences, Hopkinton, Mass. Small organic molecule libraries available for screening can be purchased, for example, from Reaction Biology Corp., Malvern, Pa.; TimTec, Newark, Del.

IV-1. Identifying Therapeutic Agents:

The differentially expressed ECT2 gene disclosed herein can also be used to identify candidate therapeutic agents for treating LC and EC. The methods of the present invention therefore involve the screening a candidate therapeutic agent to determine if the test agent can convert an expression level of the ECT2 gene that is characteristic of an LC state or an EC state to a gene expression level characteristic of a non-LC state or a non-EC state.

In the context of the instant method, a test cell population is exposed to a test agent or a plurality of test agents (sequentially or in combination) and the expression of the ECT2 gene in the cells is measured. The expression level of the gene assayed in the test cell population is compared to the expression level of the same gene in a reference cell population that is not exposed to the test agent.

An agent capable of suppressing the expression of the ECT2 gene has marked clinical benefit. Such agents can be further tested for the ability to forestall or prevent lung or esophageal carcinomal growth in animals or test subjects.

In a further embodiment, the present invention provides methods for screening candidate agents which act on the targets in the treatment of LC and/or EC. As discussed in detail above, by controlling the expression level of the ECT2 gene or the activity level of its gene product, one can control the onset and progression of LC and/or EC. Thus, candidate agents, which act on the targets in the treatment of LC and/or EC, can be identified through screening methods that use such expression and activity levels as indices of the cancerous or non-cancerous state. In the context of the present invention, such screening can include, for example, the following steps:

(a) contacting a candidate compound with a polypeptide encoded by a ECT2 polynucleotide

(b) detecting the binding activity between the polypeptide and the candidate compound; and

(c) selecting the candidate compound that binds to the polypeptide.

Alternatively, the screening methods of the present invention can include the following steps:

(a) contacting a candidate compound with a cell expressing the ECT2 gene; and

(b) selecting the candidate compound that reduces the expression level of the ECT2 gene, as compared to the expression level detected in the absence of the candidate compound.

Cells expressing the ECT2 gene include, but are not limited to, for example, cell lines established from LC or EC; such cells can be used for the above screening of the present invention.

According to the present invention, the therapeutic effect of the candidate compound on inhibiting the cell growth or a candidate compound for treating or preventing ECT2 associating disease may be evaluated. Therefore, the present invention also provides a method for screening a candidate compound that suppresses the proliferation of cancer cells, and a method for screening a candidate compound for treating or preventing ECT2 associating disease.

In the context of the present invention, such screening may include, for example, the following steps:

a) contacting a candidate compound with a cell expressing the ECT2 gene;

b) detecting the expression level of the ECT2 gene; and

c) correlating the expression level of b) with the therapeutic effect of the candidate compound.

In the present invention, the therapeutic effect may be correlated with the expression level of the ECT2 gene. For example, when the candidate compound reduces the expression level of the ECT2 gene as compared to a level detected in the absence of the test agent or compound, the candidate compound may identified or selected as the candidate agent or compound having the therapeutic effect. Alternatively, when the test agent or compound does not reduce the expression level of the ECT2 gene as compared to a level detected in the absence of the candidate compound, the candidate compound may identified as the compound having no significant therapeutic effect.

Alternatively, the screening methods of the present invention can include the following steps:

(a) contacting a candidate compound with a polypeptide encoded by a ECT2 polynucleotide;

(b) detecting the biological activity of the polypeptide of step (a); and

(c) selecting a compound that suppresses the biological activity of the polypeptide encoded by the ECT2 polynucleotide, as compared to the biological activity detected in the absence of the candidate compound.

According to the present invention, the therapeutic effect of the candidate compound on inhibiting the cell growth or a candidate compound for treating or preventing ECT2 associating disease may be evaluated. Therefore, the present invention also provides a method of screening for a candidate compound for inhibiting the cell growth or a candidate compound for treating or preventing ECT2 associating disease, using the ECT2 polypeptide or fragments thereof including the steps as follows:

a) contacting a candidate compound with the ECT2 polypeptide or a functional fragment thereof; and

b) detecting the biological activity of the polypeptide or fragment of step (a). and

c) correlating the biological activity of b) with the therapeutic effect of the test agent or compound.

In the present invention, the therapeutic effect may be correlated with the biological activity ECT2 polypeptide or a functional fragment thereof. For example, when the candidate compound suppresses or inhibits the biological activity ECT2 polypeptide or a functional fragment thereof as compared to a level detected in the absence of the candidate compound, the candidate compound may identified or selected as the candidate compound having the therapeutic effect. Alternatively, when the candidate compound does not suppress or inhibit the biological activity ECT2 polypeptide or a functional fragment thereof as compared to a level detected in the absence of the candidate compound, the candidate compound may identified as the compound having no significant therapeutic effect.

A protein for use in the screening methods of the present invention can be obtained as a recombinant protein using the known nucleotide sequence for the ECT2 gene. Based on the information regarding the ECT2 gene and its encoded protein, one skilled in the art can select any biological activity of the protein as an index for screening and any suitable measurement method to assay for the selected biological activity. Specifically, the ECT2 protein is known to have a cell proliferating activity. Therefore, the biological activity can be determined using such cell proliferating activity.

Alternatively, the screening methods of the present invention can include the following steps:

(a) contacting a candidate compound with a cell into which a vector, containing the transcriptional regulatory region of ECT2 genes and a reporter gene that is expressed under the control of the transcriptional regulatory region, has been introduced;

(b) measuring the expression or activity of said reporter gene; and

(c) selecting the candidate compound that reduces the expression or activity level of said reporter gene, as compared to the expression or activity level detected in the absence of the candidate compound.

According to the present invention, the therapeutic effect of the candidate compound on inhibiting the cell growth or a candidate compound for treating or preventing ECT2 associating disease may be evaluated. Therefore, the present invention also provides a method for screening a candidate compound that suppresses the proliferation of cancer cells, and a method for screening a candidate compound for treating or preventing ECT2 associating disease.

In the context of the present invention, such screening may include, for example, the following steps:

a) contacting a candidate compound with a cell into which a vector, composed of the transcriptional regulatory region of the ECT2 gene and a reporter gene that is expressed under the control of the transcriptional regulatory region, has been introduced;

b) detecting the expression or activity of said reporter gene; and

c) correlating the expression level of b) with the therapeutic effect of the candidate compound.

In the present invention, the therapeutic effect may be correlated with the expression or activity of said reporter gene. For example, when the candidate compound reduces the expression or activity of said reporter gene as compared to a level detected in the absence of the candidate compound, the candidate compound may identified or selected as the candidate compound having the therapeutic effect. Alternatively, when the candidate compound does not reduce the expression or activity of said reporter gene as compared to a level detected in the absence of the candidate compound, the candidate compound may identified as the agent or compound having no significant therapeutic effect.

Suitable reporter genes and host cells are well known in the art. A reporter construct suitable for the screening methods of the present invention can be prepared by using a transcriptional regulatory region of the ECT2 gene. A nucleotide segment containing the transcriptional regulatory region can be isolated from a genome library based on the nucleotide sequence information for the ECT2 gene.

The reporter construct required for the screening can be prepared by connecting reporter gene sequence to the transcriptional regulatory region of ECT2 gene. The transcriptional regulatory region of ECT2 gene herein is the region from start codon to at least 500 bp upstream, preferably 1000 bp, more preferably 5000 or 10000 bp upstream. A nucleotide segment containing the transcriptional regulatory region can be isolated from a genome library or can be propagated by PCR. Methods for identifying a transcriptional regulatory region, and also assay protocol are well known (Molecular Cloning third edition chapter 17, 2001, Cold Springs Harbor Laboratory Press).

IV-2. Selecting a Therapeutic Agent for Treating LC and/or EC:

Differences in the genetic makeup of individuals can result in differences in their relative abilities to metabolize various drugs. An agent that is metabolized in a subject to act as an anti-LC and/or EC agent can manifest itself by inducing a change in a gene expression pattern in the subject's cells from that is characteristic of a cancerous state to a gene expression pattern that is characteristic of a non-cancerous state. Accordingly, the differentially expressed ECT2 gene allows for a putative therapeutic or prophylactic inhibitor of LC and/or EC to be tested in a test cell population from a selected subject in order to determine if the agent is a suitable inhibitor of LC and/or EC in the subject.

To identify an inhibitor of LC and/or EC that is appropriate for a specific subject, a test cell population from the subject is exposed to a therapeutic agent, and the expression of the ECT2 gene is determined.

In the context of the methods of the present invention, the test cell population contains LC and/or EC cells expressing the ECT2 gene. Preferably, the test cell population includes epithelial cells. For example, a test cell population can be incubated in the presence of a candidate agent and the pattern of gene expression of the test cell population can be measured and compared to one or more reference expression profiles, e.g., an LC reference expression profile, an EC reference expression profile or normal reference expression profile, e.g., a non-LC and non-EC reference expression profile.

A decrease in the expression of the ECT2 gene in a test cell population relative to a reference cell population containing LC and/or EC indicates that the agent has therapeutic utility. Alternatively, a similarity in the expression of the ECT2 gene in the test cell population and the reference cell population indicates that the agent has alternate therapeutic utility.

In the context of the present invention, the test agent can be any compound or composition. Exemplary test agents include, but are not limited to, immunomodulatory agents (e.g., antibodies), inhibitory oligonuceotides (e.g., antisense oligonucleodies, short-inhibitory oligonucleotides and ribozymes) and small organic compounds.

IV-3. Candidate Compounds:

A compound isolated by the screening assays of the present invention may serve as a candidate for the development of drugs that inhibit the expression of the ECT2 gene or the activity of the protein encoded by the ECT2 gene and can be applied to the treatment or prevention of lung cancer and/or esophageal cancer.

Moreover, compounds in which a part of the structure of the compound inhibiting the activity of protein encoded by the ECT2 gene is converted by addition, deletion and/or replacement are also included as the compounds obtainable by the screening methods of the present invention.

A compound isolated by the screening assays of the present invention has the potential to treat or prevent cancers. Potential of these candidate compounds to treat or prevent cancers may be evaluated by second and/or further screening to identify therapeutic agent for cancers.

V. Method for Assessing the Prognosis of Lung and Esophageal Cancer:

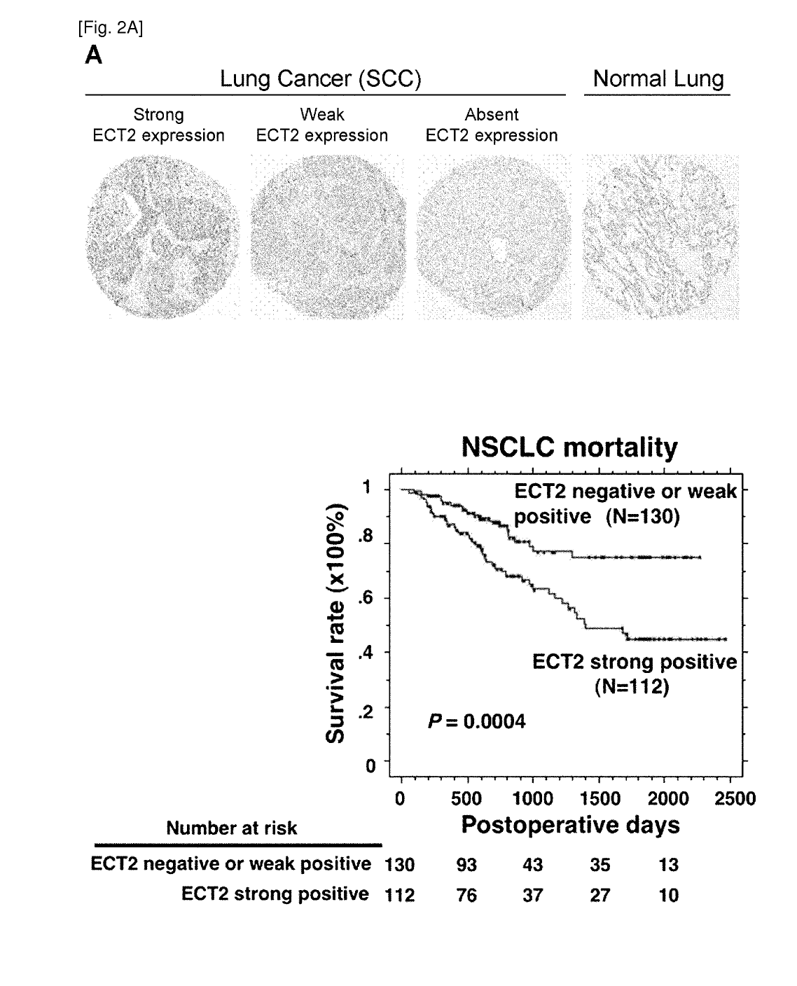

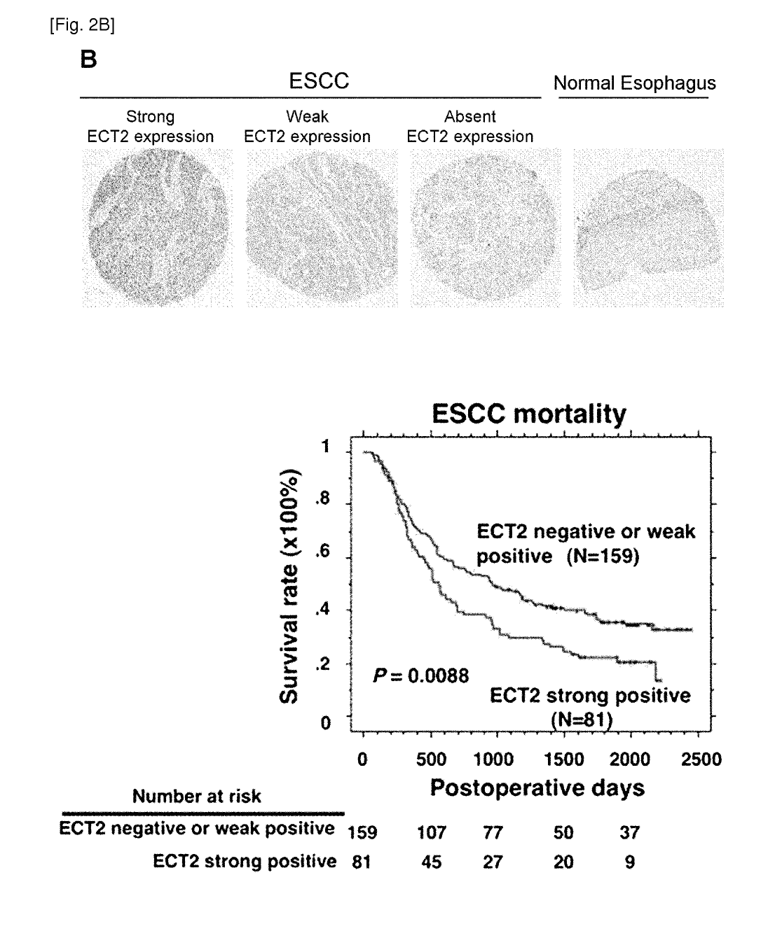

According to the present invention, it was newly discovered that ECT2 expression is significantly associated with poorer prognosis of NSCLC or ESCC patients (see FIG. 2). Thus, the present invention provides a method for assessing or determining the prognosis of a patient with lung or esophageal cancer, in particular, NSCLC or ESCC, by detecting the expression level of the ECT2 gene in a biological sample of the patient; comparing the detected expression level to a control level; and determining a increased expression level to the control level as indicative of poor prognosis (poor survival).

Herein, the term “prognosis” refers to a forecast as to the probable outcome of the disease as well as the prospect of recovery from the disease as indicated by the nature and symptoms of the case. Accordingly, a less favorable, negative, poor prognosis is defined by a lower post-treatment survival term or survival rate. Conversely, a positive, favorable, or good prognosis is defined by an elevated post-treatment survival term or survival rate.

In the context of the present invention, the phrase “assessing (or determining) the prognosis” is intended to encompass predictions and likelihood analysis of lung or esophageal cancer, progression, particularly NSCLC and ESCC recurrence, metastatic spread and disease relapse. The present method for assessing or determining prognosis is intended to be used clinically in making decisions concerning treatment modalities, including therapeutic intervention, diagnostic criteria such as disease staging, and disease monitoring and surveillance for metastasis or recurrence of neoplastic disease.

The patient-derived biological sample used for the method may be any sample derived from the subject to be assessed so long as the ECT2 gene can be detected in the sample. Preferably, the biological sample is a lung cell or an esophageal cell (a cell obtained from the lung or esophagus, respectively). Other suitable biological samples include, but are not limited to, bodily fluids such as sputum, blood, serum, or plasma. Alternatively, the sample may be cells purified from a tissue. The biological samples may be obtained from a patient at various time points, including before, during, and/or after a treatment.

According to the present invention, it was shown that the higher the expression level of the ECT2 gene measured in the patient-derived biological sample, the poorer the prognosis for post-treatment remission, recovery, and/or survival and the higher the likelihood of poor clinical outcome. Thus, according to the present method, the “control level” used for comparison may be, for example, the expression level of the ECT2 gene detected before any kind of treatment in an individual or a population of individuals who showed good or positive prognosis of NSCLC or ESCC after the treatment, which herein will be referred to as “good prognosis control level”. Alternatively, the “control level” may be, for example, the expression level of the ECT2 gene detected before any kind of treatment in an individual or a population of individuals who showed poor or negative prognosis of NSCLC or ESCC after the treatment, which herein will be referred to as “poor prognosis control level”. The “control level” is a single expression pattern derived from a single reference population or from a plurality reference population. Thus, the control level may be determined based on the expression level of the ECT2 gene detected before any kind of treatment in a patient of NSCLC or ESCC, or a population of the patients whose disease state (good or poor prognosis) is known. It is preferred, to use the standard value of the expression levels of the ECT2 gene in a patient group with a known disease state. The standard value may be obtained by any method known in the art. For example, a range of mean+/−2 S.D. or mean+/−3 S.D. may be used as standard value.

The control level may be determined at the same time with the test biological sample by using a sample(s) previously collected and stored before any kind of treatment from lung or esophageal cancer patient(s) (control or control group) whose disease state (good prognosis or poor prognosis) are known.

Alternatively, the control level may be determined by a statistical method based on the results obtained by analyzing the expression level of the ECT2 gene in samples previously collected and stored from a control group. Furthermore, the control level can be a database of expression patterns from previously tested cells. Moreover, according to an aspect of the present invention, the expression level of the ECT2 gene in a biological sample may be compared to multiple control levels, which control levels are determined from multiple reference samples. It is preferred to use a control level determined from a reference sample derived from a tissue type similar to that of the patient-derived biological sample.

According to the present invention, a similarity in the expression level of the ECT2 gene to the good prognosis control level indicates a more favorable prognosis of the patient and an increase in the expression level to the good prognosis control level indicates less favorable, poorer prognosis for post-treatment remission, recovery, survival, and/or clinical outcome. On the other hand, a decrease in the expression level of the ECT2 gene to the poor prognosis control level indicates a more favorable prognosis of the patient and a similarity in the expression level to the poor prognosis control level indicates less favorable, poorer prognosis for post-treatment remission, recovery, survival, and/or clinical outcome.

Alternatively, the present invention provides a method for detecting cancer cells in a subject-derived lung or esophageal tissue sample, said method comprising the step of determining the expression level of the ECT2 gene in a subject-derived biological sample, wherein an increase in said expression level as compared to a normal control level of said gene indicates the presence or suspicion of cancer cells in the tissue.

Such result may be combined with additional information to assist a doctor, nurse, or other healthcare practitioner in diagnosing a subject as afflicted with the disease. Alternatively, the present invention may provide a doctor with useful information to diagnose a subject as afflicted with the disease. For example, according to the present invention, when there is doubt regarding the presence of cancer cells in the tissue obtained from a subject, clinical decisions can be reached by considering the expression level of the ECT2 gene, plus a different aspect of the disease including tissue pathology, levels of known tumor marker(s) in blood, and clinical course of the subject, etc. For example, some well-known diagnostic lung tumor markers in blood are IAP, ACT, BFP, CA19-9, CA50, CA72-4, CA130, CEA, KMO-1, NSE, SCC, SP1, Span-1, TPA, CSLEX, SLX, STN and CYFRA. Alternatively, diagnostic esophageal tumor markers in blood such as CEA, DUPAN-2, IAP, NSE, SCC, SLX and Span-1 are also well known. Namely, in this particular embodiment of the present invention, the outcome of the gene expression analysis serves as an intermediate result for further diagnosis of a subject's disease state.

In another embodiment, the present invention provides a method for detecting a diagnostic marker of cancer, said method comprising the step of detecting the expression of the ECT2 gene in a subject-derived biological sample as a diagnostic marker of lung or esophageal cancer.

An expression level of the ECT2 gene in a biological sample can be considered altered when the expression level differs from the control level by more than 1.0, 1.5, 2.0, 5.0, 10.0, or more fold. Alternatively, an expression level of the ECT2 gene in a biological sample can be considered altered, when the expression level is increased or decreased to the control level at least 10%, 20%, 30%, 40%, 50%, 60%, 80%, 90%, or more.

The difference in the expression level between the test biological sample and the control level can be normalized to a control, e.g., housekeeping gene. For example, polynucleotides whose expression levels are known not to differ between the cancerous and non-cancerous cells, including those coding for beta-actin, glyceraldehyde 3-phosphate dehydrogenase, and ribosomal protein P1, may be used to normalize the expression levels of the ECT2 gene.

The expression level may be determined by detecting the gene transcript in the patient-derived biological sample using techniques well known in the art. The gene transcripts detected by the present method include both the transcription and translation products, such as mRNA and protein.

For instance, the transcription product of the ECT2 gene can be detected by hybridization, e.g., Northern blot hybridization analyses, that use an ECT2 gene probe to the gene transcript. The detection may be carried out on a chip or an array. The use of an array is preferable for detecting the expression level of a plurality of genes including the ECT2 gene. As another example, amplification-based detection methods, such as reverse-transcription based polymerase chain reaction (RT-PCR) which use primers specific to the ECT2 gene may be employed for the detection (see Example). The ECT2 gene-specific probe or primers may be designed and prepared using conventional techniques by referring to the whole sequence of the ECT2 gene (SEQ ID NO: 11). For example, the primers (SEQ ID NOs: 3 and 4) used in the Example may be employed for the detection by RT-PCR, but the present invention is not restricted thereto.

Specifically, a probe or primer used for the present method hybridizes under stringent, moderately stringent, or low stringent conditions to the mRNA of the ECT2 gene. As used herein, the phrase “stringent (hybridization) conditions” refers to conditions under which a probe or primer will hybridize to its target sequence, but to no other sequences. Stringent conditions are sequence-dependent and will be different under different circumstances. Specific hybridization of longer sequences is observed at higher temperatures than shorter sequences. Generally, the temperature of a stringent condition is selected to be about 5 degrees C. lower than the thermal melting point (Tₘ) for a specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30 degrees C. for short probes or primers (e.g., 10 to 50 nucleotides) and at least about 60 degrees C. for longer probes or primers. Stringent conditions may also be achieved with the addition of destabilizing agents, such as formamide.

Alternatively, the translation product may be detected for the assessment of the present invention. For example, the quantity of the ECT2 protein may be determined. A method for determining the quantity of the protein as the translation product includes immunoassay methods that use an antibody specifically recognizing the ECT2 protein. The antibody may be monoclonal or polyclonal. Furthermore, any fragment or modification (e.g., chimeric antibody, scFv, Fab, F(ab′)₂, Fv, etc.) of the antibody may be used for the detection, so long as the fragment retains the binding ability to the ECT2 protein. Methods to prepare these kinds of antibodies for the detection of proteins are well known in the art, and any method may be employed in the present invention to prepare such antibodies and equivalents thereof.

Alternatively, the expression level of the ECT2 gene may be determined from the intensity of staining observed via immunohistochemical analysis using an antibody against ECT2 protein. Namely, the observation of strong staining indicates increased presence of the ECT2 protein and at the same time high expression level of the ECT2 gene. NSCLC or ESCC tissue can be preferably used as a test material for immunohistochemical analysis.

Moreover, in addition to the expression level of the ECT2 gene, the expression level of other lung or esophageal cell-associated genes, for example, genes known to be differentially expressed in NSCLC or ESCC, may also be determined to improve the accuracy of the assessment. Such other lung or esophageal cell-associated genes include those described in WO 2004/031413 or WO2007/013671.

The patient to be assessed for the prognosis of NSCLC or ESCC according to the method is preferably a mammal and includes human, non-human primate, mouse, rat, dog, cat, horse, and cow.

VI. Pharmaceutical Compositions for Treating or Preventing Cancers:

VI-1. Pharmaceutical Compositions Including Double-Stranded Molecule

The present invention provides compositions for treating or preventing cancers including any of the double-stranded molecules described above in item ‘I. Double-stranded molecule’ or selected by the above-described screening methods of the present invention.

A double-stranded molecule of the present invention can be adapted for use to prevent or treat cancers which overexpressing ECT2 gene, such as lung or esophageal cancers, e.g. NSCLC or ESCC. Therefore, the preferred embodiment of the present invention is the pharmaceutical composition for treating or preventing lung or esophageal cancer, which comprises a pharmaceutically effective amount of a double-stranded molecule inhibiting the expression of ECT2 gene in a cell, wherein said double-stranded molecule comprises a sense strand and an antisense strand complementary thereto, hybridized to each other to form the double-stranded molecule and targets to a nucleotide sequence selected from the group consisting of SEQ ID NOs: 1 or 2, as an active ingredient.

In one embodiment, a composition comprising one or more double-stranded molecules of the invention can be encapsulated in a delivery vehicle, e.g. liposomes, for administration to a subject, carriers and diluents and their salts, and/or can be present in pharmaceutically acceptable formulations. Methods for the delivery of nucleic acid molecules are described in Akhtar S & Juliano R L. Trends Cell Biol. 1992 May; 2(5):139-44; Delivery Strategies for Antisense Oligonucleotide Therapeutics, ed. Akhtar, 1995; Maurer N, et al., Mol Membr Biol. 1999 January-March; 16(1):129-40; Hofland & Huang. Handb Exp Pharmacol. 1999 137:165-192. It further describes the general methods for delivery of nucleic acid molecules (U.S. Pat. No. 6,395,713 and WO 199402595). These protocols can be utilized for the delivery of virtually any double-stranded molecule. Double-stranded molecules can be administered to cells by a variety of methods known to those of skill in the art, including but not restricted to, encapsulation in liposomes, by iontophoresis, or by incorporation into other vehicles, such as biodegradable polymers, hydrogels, cyclodextrins (see for example Gonzalez H, et al., Bioconjug Chem. 1999 November-December; 10(6):1068-74; WO 03/47518 and WO 03/46185), poly (lactic-co-glycolic) acid (PLGA) and PLCA microspheres (see for example U.S. Pat. No. 6,447,796 and US 2002130430), biodegradable nanocapsules, and bioadhesive microspheres, or by proteinaceous vectors (WO 200053722). In another embodiment, the nucleic acid molecules of the invention can also be formulated or complexed with polyethyleneimine and derivatives thereof, such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives. In one embodiment, the nucleic acid molecules of the invention are formulated as described in US 20030077829 (i.e. lipid-based formulations), incorporated by reference herein in its entirety.

The double-stranded molecules of the present invention can also be administered to a subject in combination with other therapeutic compounds to increase the overall therapeutic effect. The use of multiple compounds to treat an indication can increase the beneficial effects while reducing the presence of side effects.

In another embodiment, the present invention also provides the use of the double-stranded nucleic acid molecules of the present invention in manufacturing a pharmaceutical composition for use in treating a cancer expressing the ECT2 gene. For example, the present invention relates to a use of double-stranded nucleic acid molecule inhibiting the expression of a ECT2 gene in a cell, which molecule comprises a sense strand and an antisense strand complementary thereto, hybridized to each other to form the double-stranded nucleic acid molecule and targets to a sequence selected from the group consisting of SEQ ID NOs: 1 and 2, for manufacturing a pharmaceutical composition for use in treating a cancer expressing the ECT2 gene.

Alternatively, the present invention further provides a method or process for manufacturing a pharmaceutical composition for treating a cancer expressing the ECT2 gene, wherein the method or process comprises step for formulating a pharmaceutically or physiologically acceptable carrier with a double-stranded nucleic acid molecule inhibiting the expression of a ECT2 gene in a cell, which molecule comprises a sense strand and an antisense strand complementary thereto, hybridized to each other to form the double-stranded nucleic acid molecule and targets to a sequence selected from the group consisting of SEQ ID NOs: 1 and 2 as active ingredients.

In another embodiment, the present invention also provides a method or process for manufacturing a pharmaceutical composition for treating a cancer expressing the ECT2 gene, wherein the method or process comprises step for admixing an active ingredient with a pharmaceutically or physiologically acceptable carrier, wherein the active ingredient is a double-stranded nucleic acid molecule inhibiting the expression of a ECT2 gene in a cell, which molecule comprises a sense strand and an antisense strand complementary thereto, hybridized to each other to form the double-stranded nucleic acid molecule and targets to a sequence selected from the group consisting of SEQ ID NOs: 1 and 2.

In another embodiment, the present invention also provides double-stranded nucleic acid molecules for use in treating a cancer expressing the ECT2 gene. For example, double-stranded nucleic acid molecules of the present invention comprises a sense strand and an antisense strand complementary thereto, hybridized to each other to form the double-stranded nucleic acid molecule and targets to a sequence selected from the group consisting of SEQ ID NOs: 1 and 2.

VI-2. Pharmaceutical Compositions Including Antibodies

The function of a gene product of the ECT2 gene which is over-expressed in various cancers can be inhibited by administering a compound that binds to or otherwise inhibits the function of the gene products. An antibody against the ECT2 polypeptide can be mentioned as such a compound and can be used as the active ingredient of a pharmaceutical composition for treating or preventing cancer.

The present invention relates to the use of antibodies against a protein encoded by the ECT2 gene, or fragments of the antibodies. As used herein, the term “antibody” refers to item of II. Antibody. Therefore, the preferred embodiment of the present invention is a pharmaceutical composition for treating or preventing lung cancer or esophageal cancer, which composition comprises a pharmaceutically effective amount of an antibody recognizing ECT2 binding the antigen comprising a peptide having an amino acid sequence of SEQ ID NO: 8.

An antibody may be modified by conjugation with a variety of molecules, such as polyethylene glycol (PEG). The present invention includes such modified antibodies. The modified antibody can be obtained by chemically modifying an antibody. Such modification methods are conventional in the field.

Alternatively, the antibody used for the present invention may be a chimeric antibody having a variable region derived from a non-human antibody against the ECT2 polypeptide and a constant region derived from a human antibody, or a humanized antibody, composed of a complementarity determining region (CDR) derived from a non-human antibody, a frame work region (FR) and a constant region derived from a human antibody. Such antibodies can be prepared by using known technologies. Humanization can be performed by substituting rodent CDRs or CDR sequences for the corresponding sequences of a human antibody (see e.g., Verhoeyen et al., Science 1988, 239:1534-6). Accordingly, such humanized antibodies are chimeric antibodies, wherein substantially less than an intact human variable domain has been substituted by the corresponding sequence from a non-human species.

Complete human antibodies including human variable regions in addition to human framework and constant regions can also be used. Such antibodies can be produced using various techniques known in the art. For example in vitro methods involve use of recombinant libraries of human antibody fragments displayed on bacteriophage (e.g., Hoogenboom et al., J Mol Biol 1992, 227:381-8). Similarly, human antibodies can be made by introducing human immunoglobulin loci into transgenic animals, e.g., mice in which the endogenous immunoglobulin genes have been partially or completely inactivated. This approach is described, e.g., in U.S. Pat. Nos. 6,150,584; 5,545,807; 5,545,806; 5,569,825; 5,625,126; 5,633,425; and 5,661,016.

When the obtained antibody is to be administered to the human body (antibody treatment), a human antibody or a humanized antibody is preferable for reducing immunogenicity.

Antibodies obtained as above may be purified to homogeneity. For example, the separation and purification of the antibody can be performed according to separation and purification methods used for general proteins. For example, the antibody may be separated and isolated by the appropriately selected and combined use of column chromatographies, such as affinity chromatography, filter, ultrafiltration, salting-out, dialysis, SDS polyacrylamide gel electrophoresis, isoelectric focusing, and others (Antibodies: A Laboratory Manual. Ed Harlow and David Lane, Cold Spring Harbor Laboratory (1988)), but are not limited thereto. A protein A column and protein G column can be used as the affinity column. Exemplary protein A columns to be used include, for example, Hyper D, POROS, and Sepharose F.F. (Pharmacia).

Exemplary chromatography, with the exception of affinity includes, for example, ion-exchange chromatography, hydrophobic chromatography, gel filtration, reverse-phase chromatography, adsorption chromatography, and the like (Strategies for Protein Purification and Characterization: A Laboratory Course Manual. Ed Daniel R. Marshak et al., Cold Spring Harbor Laboratory Press (1996)). The chromatographic procedures can be carried out by liquid-phase chromatography, such as HPLC and FPLC.

In another embodiment, the present invention also provides the use of the antibody of the present invention in manufacturing a pharmaceutical composition for use in treating a cancer expressing the ECT2 gene.

Alternatively, the present invention further provides a method or process for manufacturing a pharmaceutical composition for treating a cancer expressing the ECT2 gene, wherein the method or process comprises step for formulating a pharmaceutically or physiologically acceptable carrier with an antibody.

In another embodiment, the present invention also provides a method or process for manufacturing a pharmaceutical composition for treating a cancer expressing the ECT2 gene, wherein the method or process comprises step for admixing an active ingredient with a pharmaceutically or physiologically acceptable carrier, wherein the active ingredient is an antibody.

In another embodiment, the present invention also provides an antibody for use in treating a cancer expressing the ECT2 gene.

Unless otherwise defined, all technical and scientific terms used herein have the same meaning as commonly understood by one of ordinary skill in the art to which this invention belongs. In case of conflict, the present specification, including definitions, will control.

Hereinafter, the present invention is described in more detail with reference to the Examples. However, the following materials, methods and examples only illustrate aspects of the invention and in no way are intended to limit the scope of the present invention. As such, methods and materials similar or equivalent to those described herein can be used in the practice or testing of the present invention.

### Examples

### I. Materials and Methods

A. Cell Lines and Tissue Samples

Fifteen human lung-cancer cell lines used in this study included five adenocarcinomas (ADCs; NCI-H1781, NCI-H1373, LC319, A549, and PC-14), five squamous-cell carcinomas (SCCs; SK-MES-1, NCI-H2170, NCI-H520, NCI-H1703, and LU61), one large-cell carcinoma (LCC; LX1), and four small-cell lung cancers (SCLCs; SBC-3, SBC-5, DMS273, and DMS114). Ten human esophageal carcinoma cell lines used in this study were as follows; 9 SCC cell lines (TE1, TE2, TE3, TE4, TE5, TE6, TE8, TE9, and TE10) and one ADC cell line (TE7) (Nishihira T. et al. J Cancer Res Clin Oncol 1993; 119: 441-49.). All cells were grown in monolayer in appropriate media supplemented with 10% fetal calf serum (FCS) and were maintained at 37° C. in humidified air with 5% CO2. Human small airway epithelial cells (SAEC) used as a normal control were grown in optimized medium (SAGM) from Cambrex Bio Science Inc.

Primary NSCLC and ESCC tissue samples from patients having no anticancer treatment before tumor resection had been obtained earlier with informed consent (Kikuchi T. et al. Oncogene 2003; 22:2192-205, Taniwaki M. et al. Int J Oncol 2006; 29:567-75, Yamabuki T. et al. Int J Oncol 2006; 28:1375-84, Kato T. et al. Cancer Res 2005; 65:5638-46.). All tumors were staged on the basis of the pTNM pathological classification of the UICC (International Union Against Cancer; Table 1) (Sobin L. and Wittekind Ch., 6th edition. New York: Wiley-Liss; 2002.). Formalin-fixed primary lung tumors and adjacent normal lung tissue samples used for immunostaining on tissue microarrays had been obtained from 242 patients (136 ADCs, 87 SCCs, 16 LCCs, 3 ASCs; 76 female and 166 male patients; median age of 63.3 with a range of 26-84 years) undergoing surgery. A total of 240 formalin-fixed primary ESCCs (21 female and 219 male patients; median age of 61.9 with a range of 41-81 years) and adjacent normal esophageal tissue samples had been obtained from patients undergoing surgery. This study and the use of all clinical materials mentioned were approved by individual institutional Ethical Committees.

B Semiquantitative RT-PCR

A total of 3-mc (micro)_{g} aliquot of mRNA from each sample was reversely transcribed to single-stranded cDNAs using random primer (Roche Diagnostics) and Superscript II (Invitrogen). Semiquantitative RT-PCR experiments were carried out with the following sets of synthesized primers specific for human ECT2 or with beta-actin (ACTB)-specific primers as an internal control: ECT2,

| | |
|---|---|
| 5′-GCGTTTTCAAGATCTAGCATGTG-3′ | (SEQ ID NO.: 3) |
| and | |
| 5′-CAATTTTCCCATGGTCTTATCC-3′, | (SEQ ID NO.: 4) |
| ACTB, | |
| 5′-GAGGTGATAGCATTGCTTTCG-3′ | (SEQ ID NO.: 5) |
| and | |
| 5′-CAAGTCAGTGTACAGGTAAGC-3′. | (SEQ ID NO.: 6) |

PCR reactions were optimized for the number of cycles to ensure product intensity to be within the linear phase of amplification.

C Northern-Blot Analysis

Human multiple-tissue blots covering 23 tissues (BD Bioscience) were hybridized with an [alpha-32P]-dCTP-labeled, 719-bp PCR product of ECT2 that was prepared as a probe using primers

| | |
|---|---|
| 5′-TGGTGAAAGCTGGAAGGAAG-3′ | (SEQ ID NO.: 7) |
| and | |
| 5′-CAATTTTCCCATGGTCTTATCC-3′. | (SEQ ID NO.: 4) |

Prehybridization, hybridization, and washing were performed following manufacturer's recommendation. The blots were autoradiographed with intensifying screens at −80 degrees C. for 7 days.

D Anti-ECT2 Antibodies

Plasmids expressing COOH-terminal portion of ECT2 (codons 703-883) (SEQ ID NO.:8) that contained His-tagged epitopes at their NH2-terminals were prepared using pET28 vector (Novagen). The recombinant proteins were expressed in Escherichia coli, BL21 codon-plus strain (Stratagene), and purified using Ni-NTA Superflow (QIAGEN) according to the supplier's protocol. The protein was inoculated into rabbits; the immune sera were purified on affinity columns according to standard methodology. The affinity-purified anti-ECT2 polyclonal antibodies were used for western blotting and immunostaining. It was confirmed that the antibody was specific to ECT2 on western blots using lysates from cell lines that had been transfected with ECT2 expression vector and those from lung and esophageal cancer cell lines, either of which expressed ECT2 endogenously or not.

E Western Blotting

Tumor cells were lysed in lysis buffer; 50 mM Tris-HCl (pH 8.0), 150 mM NaCl, 0.5% NP40, 0.5% sodium deoxycholate, and Protease Inhibitor Cocktail Set III (Calbiochem). The protein content of each lysate was determined by a Bio-Rad protein assay (Bio-Rad) with bovine serum albumin (BSA) as a standard. Ten micrograms of each lysate were resolved on 10-12% denaturing polyacrylamide gels (with 3% poly-acrylamide stacking gel) and transferred electrophoretically to a nitrocellulose membrane (GE Healthcare Bio-sciences). After blocking with 5% non-fat dry milk in TBST, the membrane was incubated with a rabbit polyclonal anti-human ECT2 antibody (generated to recombinant ECT2; please see above) for 1 hour at room temperature. Immunoreactive proteins were incubated with horseradish peroxidase-conjugated secondary antibodies (GE Healthcare Bio-sciences) for 1 hour at room temperature. After washing with TBST, the reactants were developed using the enhanced chemiluminescence kit (GE Healthcare Bio-sciences).

F Immunohistochemistry and Tissue Microarray

To investigate clinicopathological significance of the ECT2 protein in clinical samples that had been formalin-fixed and embedded in paraffin blocks, the sections was stained using ENVISION+Kit/HRP (DakoCytomation) in the following manner. For antigen retrieval, slides were immersed in Target Retrieval Solution High pH (DakoCytomation) and boiled at 108 degrees C. for 15 min in an autoclave. 3.3 mcg/ml of a rabbit polyclonal anti-human ECT2 antibody (generated to recombinant ECT2; please see above) was added to each slide after blocking of endogenous peroxidase and proteins, and the sections were incubated with horseradish peroxidase-labeled anti-rabbit IgG (Histofine Simple Stain MAX PO (G), Nichirei) as the secondary antibody. Substrate-chromogen was added, and the specimens were counterstained with hematoxylin.

Tumor tissue microarrays were constructed with 242 formalin-fixed primary NSCLCs and 240 primary ESCCs, each of which had been obtained by a single institutional group (please see above) with an identical protocol to collect, fix, and preserve the tissues after resection (Chin S. et al. Mol Pathol 2003; 56:275-79, Callagy G. et al. Diagn Mol Pathol 2003; 12:27-34, Callagy G. et al. J Pathol 2005; 205:388-96.). Considering the histological heterogeneity of individual tumors, tissue area for sampling was selected based on visual alignment with the corresponding H&E-stained section on a slide. Three, four, or five tissue cores (diameter, 0.6 mm; depth, 3-4 mm) taken from a donor tumor block were placed into a recipient paraffin block with a tissue microarrayer (Beecher Instruments). A core of normal tissue was punched from each case, and 5-mcm sections of the resulting microarray block were used for immunohistochemical analysis. Three independent investigators semiquantitatively assessed ECT2 positivity without prior knowledge of clinicopathological data. Since the intensity of staining within each tumor tissue core was mostly homogenous, the intensity of ECT2 staining was semiquantitatively evaluated using following criteria: strong positive (scored as 2+), dark brown staining in more than 50% of tumor cells completely obscuring nucleus and cytoplasm; weak positive (1+), any lesser degree of brown staining appreciable in tumor cell nucleus and cytoplasm; absent (scored as 0), no appreciable staining in tumor cells. Cases were accepted as strongly positive only if reviewers independently defined them as such.

G Statistical Analysis

Statistical analyses were performed using the StatView statistical program (SaS). Strong ECT2 immunoreactivity was assessed for association with clinicopathologic variables such as age, gender, pathological TNM stage, and histological type using the Fisher's exact test. Tumor-specific survival curves were calculated from the date of surgery to the time of death related to NSCLC or ESCC, or to the last follow-up observation. Kaplan-Meier curves were calculated for each relevant variable and for ECT2 expression; differences in survival times among patient subgroups were analyzed using the log-rank test. Univariate and multivariate analyses were performed with the Cox proportional-hazard regression model to determine associations between clinicopathological variables and cancer-related mortality. First, the present inventors analyzed associations between death and possible prognostic factors including age, gender, histology, pT-classification, and pN-classification taking into consideration one factor at a time. Second, multivariate Cox analysis was applied on backward (stepwise) procedures that always forced strong ECT2 expression into the model, along with any and all variables that satisfied an entry level of a P-value less than 0.05. As the model continued to add factors, independent factors did not exceed an exit level of P<0.05.

H RNA Interference Assay

To evaluate the biological functions of ECT2 in lung and esophageal cancer cells, small interfering RNA (siRNA) duplexes were used against the target genes (Dharmacon). The target sequences of the synthetic oligonucleotides for RNAi were as follows: control 1 (Luciferase (LUC): Photinus pyralis luciferase gene),

| | |
|---|---|
| 5′-CGUACGCGGAAUACUUCGA-3′ | (SEQ ID NO.: 9); |

control 2 (Scramble (SCR): chloroplast Euglena gracilis gene coding for 5S and 16S rRNAs),

| | |
|---|---|
| 5′-GCGCGCUUUGUAGGAUUCG-3′; | (SEQ ID NO.: 10) |
| si-ECT2-#1, | |
| 5′-GAUAAAGGAUGAUCUUGAA-3′; | (SEQ ID NO.: 1) |
| si-ECT2-#2, | |
| 5′-CAGAGGAGAUUAAGACUAU-3′. | (SEQ ID NO.: 2) |

A lung cancer cell line A549 and an esophageal cancer cell line TE9 were plated onto 10-cm dishes (1.5×10⁶ cells per dish), and transfected with either of the siRNA oligonucleotides (100 nM), using 24 mcl of Lipofectamine 2000 (Invitrogen), according to the manufacturers' instructions. After 7 days of incubation, these cells were stained by Giemsa solution to assess colony formation, and cell numbers were assessed by 3-(4,5-dimethylthiazol-2-yl)-2,5-diphenyltetrazolium bromide (MTT) assay.

I Flow Cytometry

Cells transfected with siRNA oligonucleotides were plated at densities of 5×10⁵ cells/100 mm dish. Cells were trypsinized two or three days after transfection, collected in PBS, and fixed in 70% cold ethanol for 30 minutes. After treatment with 100 mcg/ml RNase (Sigma-Aldrich), the cells were stained with 50 mcg/ml propidium iodide (Sigma-Aldrich) in PBS. Flow cytometry was done on a Becton Dickinson FACScan and analyzed by ModFit software (Verity Software House, Inc.). The cells selected from at least 20,000 ungated cells were analyzed for DNA content.

J Matrigel Invasion Assay

NIH3T3 and COS-7 cells transfected either with p3XFLAG-tagged (C-terminal) plasmid designed to express ECT2 or with mock plasmid were grown to near confluence in DMEM containing 10% FCS. The cells were harvested by trypsinization, washed in DMEM without addition of serum or proteinase inhibitor, and suspended in DMEM at concentration of 1×10⁵ cells/ml. Before preparing the cell suspension, the dried layer of Matrigel matrix (Becton Dickinson Labware) was rehydrated with DMEM for 2 hours at room temperature. DMEM (0.75 ml) containing 10% FCS was added to each lower chamber in 24-well Matrigel invasion chambers, and 0.5 ml (5×10⁴ cells) of cell suspension was added to each insert of the upper chamber. The plates of inserts were incubated for 24 hours at 37 degrees C. Then the chambers were processed; cells invading through the Matrigel were fixed and stained by Giemsa as directed by the supplier (Becton Dickinson Labware).

### II. Results

A. ECT2 Expression in Lung and Esophageal Cancers and Normal Tissues

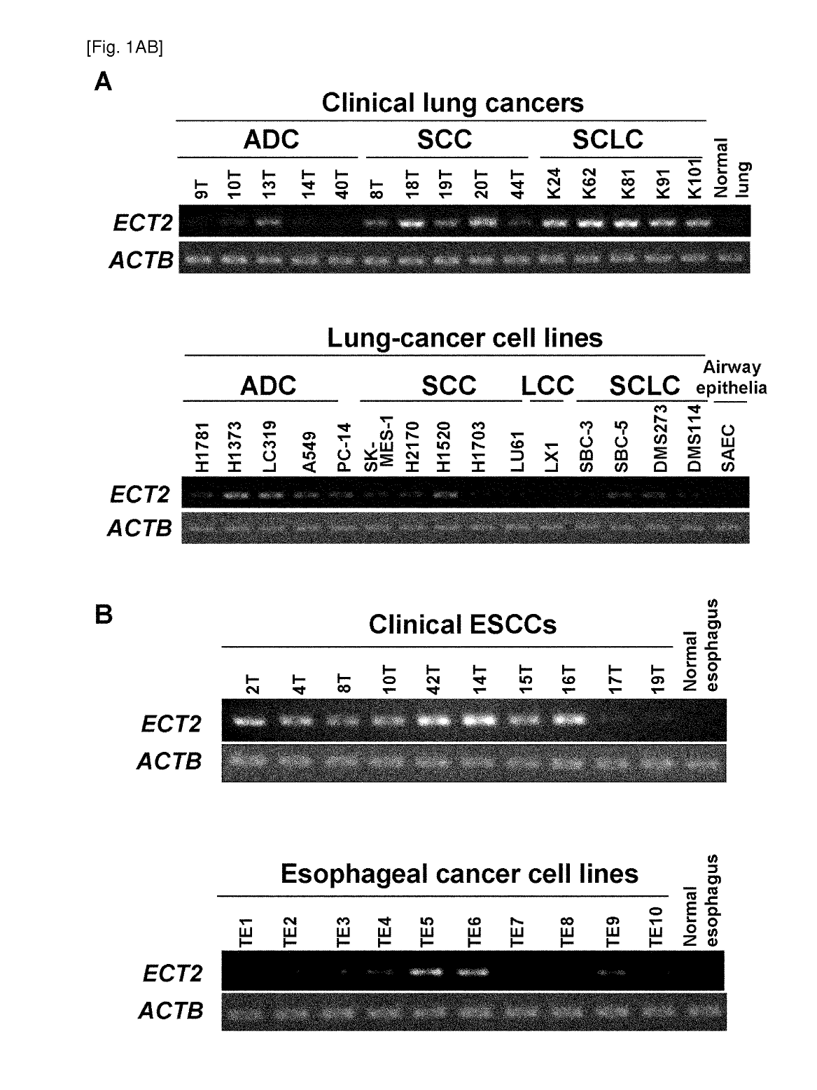

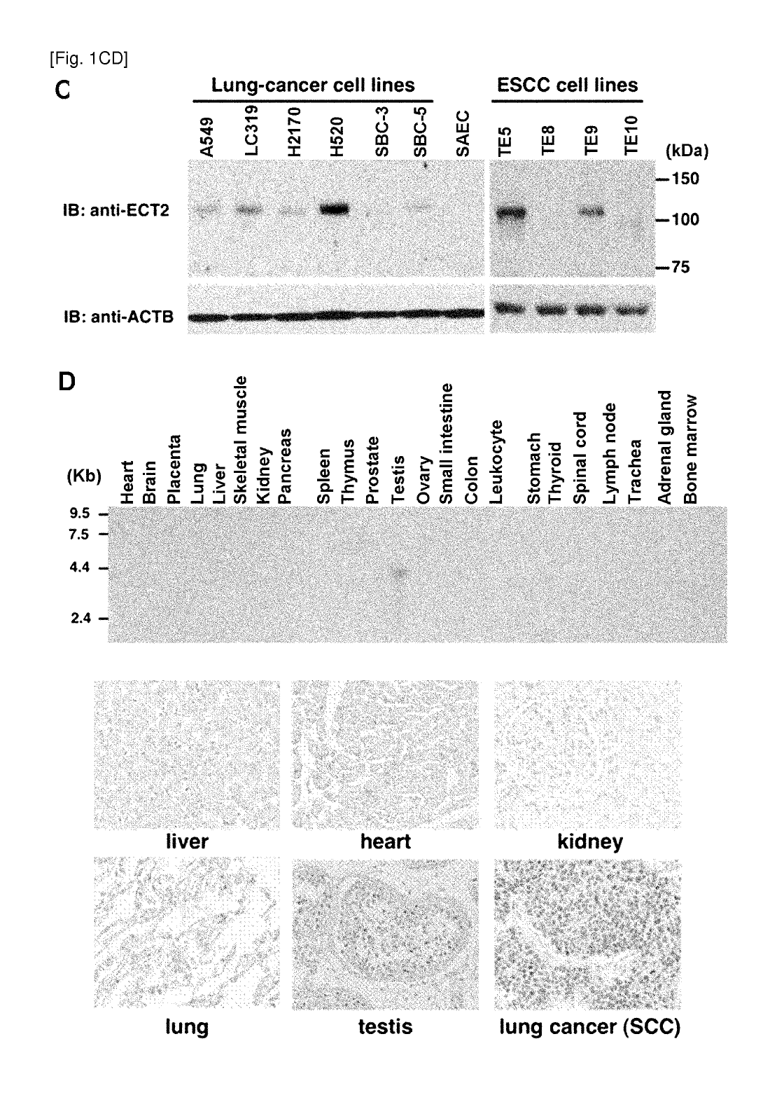

The present inventors previously performed the genome-wide expression profile analysis of 101 lung carcinomas (86 NSCLCs or 15 SCLCs) and 19 ESCCs using cDNA microarray consisting of 27,648 genes or ESTs (Kikuchi T. et al. Oncogene 2003; 22:2192-205, Kakiuchi S. et al. Mol Cancer Res 2003; 1:485-99, Kakiuchi S. et al. Hum Mol Genet. 2004; 13:3029-43, Kikuchi T. et al. Int J Oncol 2006; 28:799-805, Taniwaki M. et al. Int J Oncol 2006; 29:567-75, Yamabuki T. et al. Int J Oncol 2006; 28:1375-84. WO 2004/031413, WO2007/013671), and identified elevated expression (3-fold or higher) of ECT2 transcript in cancer cells in the great majority of the lung and esophageal cancer samples examined. The inventors confirmed its over-expression by means of semiquantitative RT-PCR experiments in 12 of 15 lung cancer tissues, in 10 of 15 lung-cancer cell lines, in 8 of 10 ESCC tissues, and in 4 of 10 ESCC cell lines examined (FIGS. 1A and 1B). The inventors subsequently generated rabbit polyclonal antibodies specific for human ECT2 and confirmed by western-blot analysis the overexpression of ECT2 protein in 5 of 6 lung cancer cell lines and 2 of 4 ESCC cell lines (FIG. 1C). Northern blot analysis using an ECT2 cDNA as a probe identified a 4.3-kb transcript only in testis among 23 normal human tissues examined (FIG. 1D, top panel). The present inventors subsequently examined expression of ECT2 protein in five normal tissues (liver, heart, kidney, lung, and testis) as well as lung cancers using anti-ECT2 antibody, and found that it was hardly detectable in the former four tissues while positive ECT2 staining appeared in the nucleus and cytoplasm of testis and lung cancer cells (FIG. 1D, bottom panels).

Northern blot analysis using an ECT2 cDNA as a probe identified a 4.3-kb transcript only in testis among 23 normal human tissues examined (FIG. 1D, top panel). The present inventors subsequently examined expression of ECT2 protein in five normal tissues (liver, heart, kidney, lung, and testis) as well as lung cancers using anti-ECT2 antibody, and found that it was hardly detectable in the former four tissues while positive ECT2 staining appeared in the nucleus and cytoplasm of testis and lung cancer cells (FIG. 1D, bottom panels).

B Association of ECT2 Overexpression with Poor Prognosis for NSCLC and ESCC Patients

To investigate the biological and clinicopathological significance of ECT2 in pulmonary and esophageal carcinogenesis, the inventors carried out immunohisto-chemical staining on tissue microarray containing tissue sections from 242 NSCLC and 240 ESCC cases who had undergone surgical resection. ECT2 staining with the anti-ECT2 polyclonal antibody was mainly observed at nucleus and cytoplasm of lung tumor cells, but not detected in their surrounding normal lung cells (FIG. 2A, top panels). The inventors classified ECT2 expression levels on the tissue array ranging from absent (scored as 0) to weak/strong positive (scored as 1+˜2+). Of the 242 NSCLCs, ECT2 was strongly stained in 112 cases (46%; score 2+), weakly stained in 91 cases (38%; score 1+), and not stained in 39 cases (16%: score 0) (details are shown in Table 1A). The present inventors next examined a correlation of ECT2 expression levels (strong positive versus weak positive/absent) with various clinicopathologic variables, and found that strong expression of ECT2 in NSCLCs was significantly associated with non-ADC histology (P=0.0389, Fisher's exact test; Table 1A) and with tumor-specific 5-year survival after the resection of primary tumors (P=0.0004, log-rank test; FIG. 2A, bottom panel). The present inventors also applied univariate analysis to evaluate associations between patient prognosis and several factors including age (>65 versus <65), gender (male versus female), histology (non-ADC versus ADC), pT stage (tumor size; T2-T3 versus T1), pN stage (lymph-node metastasis; N1-N2 versus N0), and ECT2 expression (score 2+ versus 0, 1+). All those parameters were significantly associated with poor prognosis (Table 1B). Multivariate analysis using the Cox proportional-hazard model indicated that pT stage, pN stage, age, and strong ECT2 staining were independent prognostic factors for NSCLC (Table 1B).

TABLE 1A
Association between ECT2-positivity in NSCLC tissues
and patients' characteristics (n = 242)

| | | ECT2 strong | ECT2 weak | ECT2 | P-value |
|---|---|---|---|---|---|
| | Total | positive | positive | absent | strong vs |
| | n = 242 | n = 112 | n = 91 | n = 39 | weak/absent |
| Age (years) | | | | | |
| <65 | 118 | 52 | 44 | 22 | 0.5006 |
| >=65 | 124 | 60 | 47 | 17 | |
| Gender | | | | | |
| Female | 76 | 32 | 35 | 9 | 0.378 |
| Male | 166 | 80 | 56 | 30 | |
| Histology | | | | | |
| *ADC | 136 | 55 | 60 | 21 | 0.0389⁺ |
| Non-ADC | 106 | 57 | 31 | 18 | |
| pT factor | | | | | |
| T1 | 100 | 48 | 41 | 11 | 0.6527 |
| T2 + T3 | 142 | 64 | 50 | 28 | |
| pN factor | | | | | |
| N0 | 183 | 87 | 67 | 29 | 0.4887 |
| N1 + N2 | 59 | 25 | 24 | 10 | |

*ADC: adenocarcinoma
⁺P < 0.05 (Fisher's exact test)

TABLE 1B
Cox's proportional hazards model analysis of prognostic factors in patients with NSCLC

| | | | | |
|---|---|---|---|---|
| Variables | Hazards ratio | 95% Cl | Unfavorable/Favorable | P-value |

Univariate analysis

| | | | | |
|---|---|---|---|---|
| ECT2 | 2.477 | 1.470-4.175 | Strong(+)/Weak(+) or (−) | 0.0007⁺ |
| Age ( years ) | 2.318 | 1.386-3.876 | >=65/65> | 0.0013⁺ |
| Gender | 2.040 | 1.126-3.695 | Male/Female | 0.0186⁺ |
| Histlogy | 2.484 | 1.490-4.139 | Non-ADC/ADC | 0.0005⁺ |
| pT factor | 3.766 | 2.006-7.068 | T2 + T3/T1 | <0.0001⁺ |
| pN factor | 3.715 | 2.266-6.086 | N1 + N2/N0 | <0.0001⁺ |

Multivariate analysis

| | | | | |
|---|---|---|---|---|
| ECT2 | 2.672 | 1.536-4.648 | Strong(+)/Weak(+) or (−) | 0.0005⁺ |
| Age ( years ) | 1.852 | 1.087-3.154 | >=65/65> | 0.0234⁺ |
| Gender | 1.175 | 0.590-2.340 | Male/Female | 0.6471 |
| Histology | 1.265 | 0.678-2.360 | Non-ADC/ADC | 0.4603 |
| pT factor | 2.519 | 1.271-4.990 | T2 + T3/T1 | 0.0081⁺ |
| pN factor | 3.339 | 2.004-5.562 | N1 + N2/N0 | <0.0001⁺ |

⁺P < 0.05

Of the 240 ESCC cases examined, ECT2 was strongly stained in 81 cases (34%; score 2+), weakly stained in 135 cases (56%; score 1+) and not stained in 24 cases (10%; score 0) (FIG. 2B, top panels) (details are shown in Table 2A). The present inventors found a significant correlation of strong ECT2 positivity (score 2+) with pT stage (higher in deeper tumor invasion cases; P=0.0124) and pN stage (higher in lymph node metastasis positive cases; P=0.0442 by Fisher's exact test; Table 2A). ESCC patients whose tumors showed strong ECT2 expression revealed shorter tumor-specific survival periods compared with those with absent/weak ECT2 expression (P=0.0088 by log-rank test; FIG. 2B, bottom panel). Univariate analysis evaluating associations between ESCC prognosis and several factors including age (≧65 versus <65), gender (male versus female), pT stage (tumor depth; T2, T3 versus T1), pN stage (N1 versus N0), and ECT2 expression (score 2+14 versus 0, 1+) revealed that all of those parameters except the age were significantly associated with poor prognosis (Table 2B). In multivariate analysis, strong ECT2 expression did not reach the statistically significant level as an independent prognostic factor for surgically-treated ESCC patients enrolled in this study (P=0.1872), while pT and pN stages as well as gender did so, suggesting the relevance of ECT2 expression to these clinicopathological factors in esophageal cancer (Table 2B).

TABLE 2A
Association between ECT2-positivity in ESCC tissues and
patients' characteristics (n = 240)

| | | ECT2 strong | ECT2 weak | ECT2 | P-value |
|---|---|---|---|---|---|
| | Total | positive | positive | absent | strong vs |
| | n = 240 | n = 81 | n = 135 | n = 24 | weak/absent |
| Age (years) | | | | | |
| <65 | 144 | 51 | 80 | 13 | 0.5036 |
| >=65 | 96 | 30 | 55 | 11 | |
| Gender | | | | | |
| Female | 21 | 6 | 12 | 3 | 0.5993 |
| Male | 219 | 75 | 123 | 21 | |
| pT factor | | | | | |
| T1 | 52 | 10 | 34 | 8 | 0.0124⁺ |
| T2 + T3 | 188 | 71 | 101 | 16 | |
| pN factor | | | | | |
| N0 | 67 | 16 | 44 | 7 | 0.0442⁺ |
| N1 | 173 | 65 | 91 | 17 | |

⁺P < 0.05 (Fisher's exact test)

TABLE 2B
Cox's proportional hazards model analysis of prognostic factors in patients with ESCCs

| | | | | |
|---|---|---|---|---|
| Variables | Hazards ratio | 95% CI | Unfavorable/Favorable | P-value |

Univariate analysis

| | | | | |
|---|---|---|---|---|
| ECT2 | 1.514 | 1.108-2.070 | Strong(+)/Weak(+) or (−) | 0.0093⁺ |
| Age ( years ) | 1.054 | 0.772-1.439 | >=65/65> | 0.7395 |
| Gender | 2.843 | 1.396-5.791 | Male/Female | 0.0040⁺ |
| pT factor | 2.446 | 1.585-3.775 | T2 + T3/T1 | <0.0001⁺ |
| pN factor | 3.119 | 2.073-4.694 | N1/N0 | <0.0001⁺ |

Multivariate analysis

| | | | | |
|---|---|---|---|---|
| ECT2 | 1.237 | 0.902-1.698 | Strong(+)/Weak(+) or (−) | 0.1872 |
| Gender | 2.847 | 1.396-5.803 | Male/Female | 0.0040⁺ |
| pT factor | 1.799 | 1.145-2.828 | T2 + T3/T1 | 0.0109⁺ |
| pN factor | 2.551 | 1.671-3.896 | N1/N0 | <0.0001⁺ |

⁺P < 0.05

C. Inhibition of Growth of Cancer Cells by Small Interfering RNA for ECT2

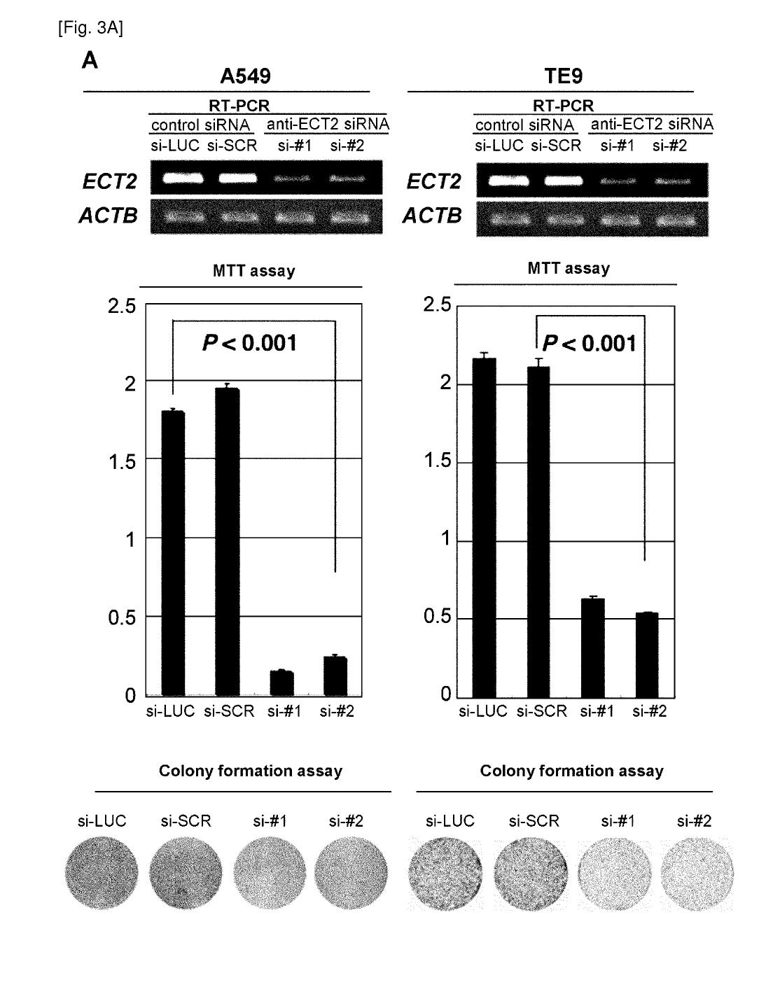

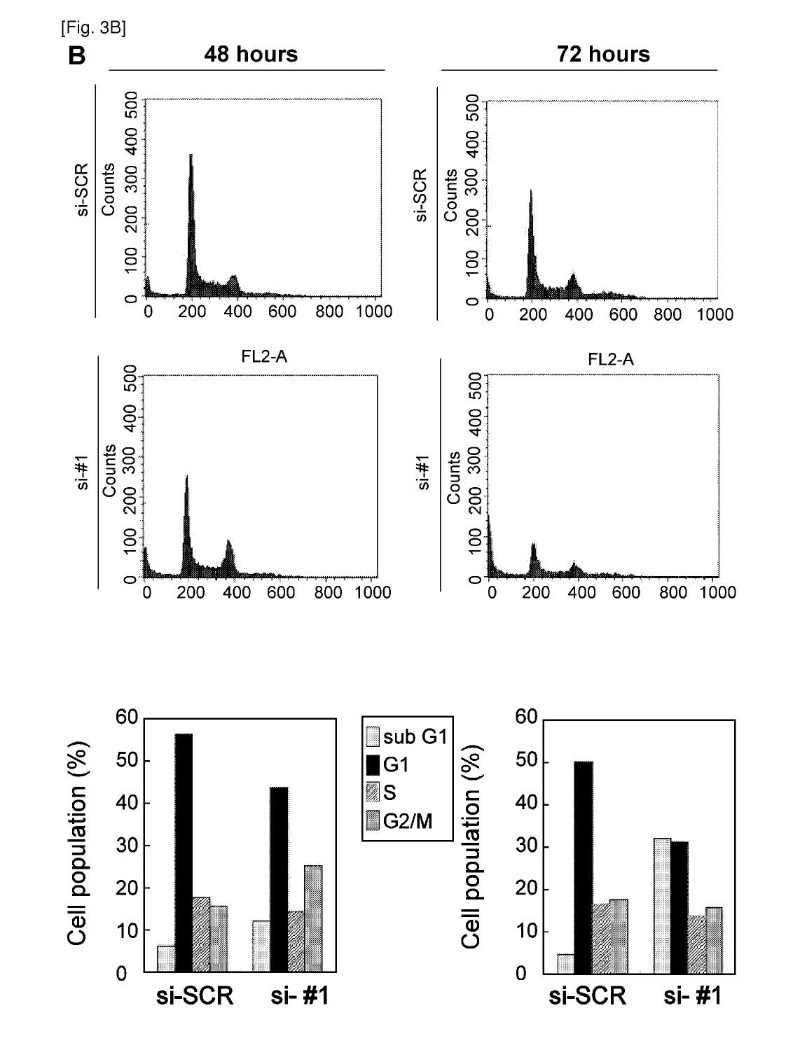

To assess whether ECT2 is essential for growth or survival of lung and esophageal cancer cells, the inventors transfected synthetic oligonucleotide siRNAs against ECT2 into A549 and TE9 cells in which ECT2 was endogenously overexpressed. The levels of ECT2 in the cells transfected with si-ECT2-#1 or -#2 were significantly decreased in comparison with those transfected with either control siRNAs (FIG. 3A, top panels). MTT and colony-formation assays revealed a drastic reduction in the number of cells transfected with si-ECT2-#1 or -#2 (FIG. 3A, middle and bottom panels). To clarify the mechanism of tumor suppression by siRNAs against ECT2, the inventors performed flow cytometric analysis of the tumor cells transfected with these siRNAs, and found a significant increase of the cells at the G2/M phase at 48 hours and a subsequent increase of the cells of sub-G1 fraction at 72 hours after the treatment (FIG. 3B).

D. Activation of Mammalian Cellular Invasion by ECT2

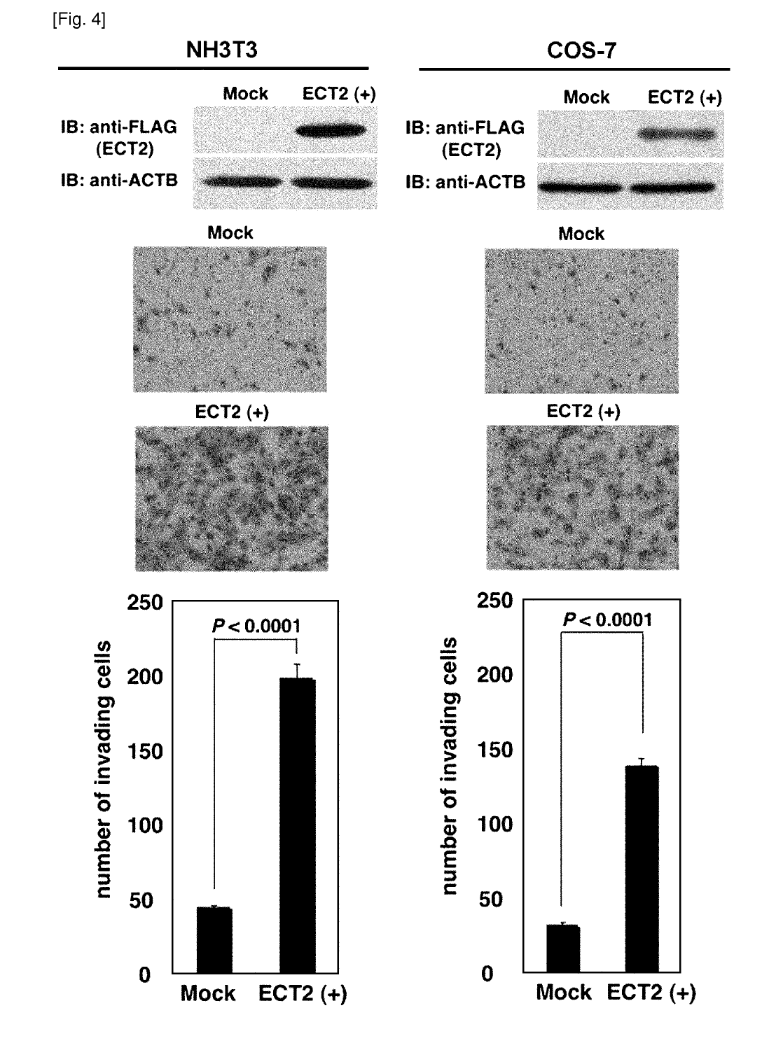

Since ECT2 is a guanine nucleotide exchange factor (GEF) for Rho GTPases which may be associated with cell motility, and the immunohistochemical analysis on tissue microarray had indicated that lung and esophageal cancer patients with strong ECT2-positive tumors showed shorter cancer-specific survival period than those with ECT2-weak positive/negative tumors, the present inventors examined a possible role of ECT2 in cellular invasion by Matrigel assays using two mammalian cells (NIH3T3 and COS-7). Transfection of ECT2 cDNA into either of the cells significantly enhanced their invasive activity through Matrigel (FIG. 4). This result also suggested that ECT2 could contribute to the highly malignant phenotype of cells.

### III. Discussion of the Results

Aerodigestive tract cancer including carcinomas of the lung, esophagus, oral cavity, pharynx, and larynx accounts for one-third of all cancer deaths in the United States and is the most common cancer in some areas of the world (Daigo Y. and Nakamura Y. Gen Thorac Cardiovasc Surg 2008; 56:43-53.). Despite the use of modern surgical techniques combined with various adjuvant treatment modalities such as radiotherapy and chemotherapy, the overall 5-year survival rate of ESCC patients remains at around 40%, and that of lung cancer patients is only 15% (Parkin D M. Lancet Oncol 2001; 2:533-43, Shimada H. et al. Surgery 2003; 133:486-94.). Therefore, further development of new cancer diagnostics and therapeutics by targeting specific oncogenic pathways is urgently awaited. The present inventors performed a genome-wide expression profile analysis of 101 lung cancers and 19 ESCCs after enrichment of cancer cells by laser microdissection, using a cDNA microarray containing 27,648 genes. The present inventors systematically analyzed the protein expression of candidate targets among hundreds of clinical samples on tissue microarrays, investigated loss-of-function phenotypes using RNAi systems and further defined biological functions of the proteins. Through these analyses, the inventors have identified a number of onco-proteins that were up-regulated in cancer cells, but not expressed in normal organs, and could be potentially good candidates for the development of novel diagnostic biomarkers, therapeutic drugs, and/or immunotherapy (Daigo Y. and Nakamura Y. Gen Thorac Cardiovasc Surg 2008; 56:43-53, Mizukami Y. et al. Cancer Sci 2008; 99:1448-54.). In this study, it is disclosed for the first time that ECT2 encoding a guanine nucleotide exchange factor (GEF) for Rho GTPases, is frequently trans-activated in the majority of lung and esophageal cancer samples, and that its gene products play indispensable roles in the growth/invasion of the cancer cells.

The small Rho GTPase is known to play important roles in essential cellular processes such as the regulation of actin cytoskeleton, gene transcription, cell motility, cell adhesion, and cytokinesis (Etienne S. et al. J Immunol 1998; 161: 5755-61, Kaibuchi K. Prog Mol Subcell Biol 1999; 22: 23-38.). ECT2 contains a Dbl homology (DH) domain in tandem with a pleckstrin homology (PH) domain, and catalyzes guanine nucleotide exchange on the small GTP-binding protein, such as RhoA and Cdc42 (Miki T Methods Enzymol 1995; 256:90-8, Das B. et al. J Biol Chem 2000; 275:15074-81.). ECT2 expression is directly regulated by E2Fs (Eguchi T. et al. Oncogene 2007; 26: 509-20.), and ECT2 protein is phosphorylated at Thr341 by CDK1 during G2/M phase, resulting in increase of the GEF activity and regulation of cytokinesis (Hara T. et al. Oncogene 2006; 25: 566-78.). It has been proposed that late mitotic Plk1 activity promotes recruitment of ECT2 to the central spindle, triggering the initiation of cytokinesis and contributing to cleavage plane specification in human cells (Petronczki M. et al. Dev Cell 2007; 12: 713-25.). In this study, it was demonstrated that the treatment of cancer cells with specific siRNA for ECT2 results in inhibition of cancer-cell growth through G2/M arrest at 48 hours after the siRNA transfection and subsequent apoptosis at 72 hours. Additional evidence also shows the significance of ECT2 in human carcinogenesis. The expression of ECT2 resulted in the significant promotion of the cellular invasion. Moreover, clinicopathological evidence obtained through tissue-microarray experiments indicates that NSCLC or ESCC patients with ECT2-positive tumors have shorter cancer-specific survival periods than those with ECT2-negative tumors. Although the exact molecular mechanisms underlying increased ECT2 expression levels in lung and esophageal cancer cells have not been elucidated, the results obtained by in vitro and in vivo assays demonstrate that ECT2 is likely to be an important growth factor and might be associated with a highly malignant phenotype of cancer cells. Based on the evidence provided here, ECT2 can now be classified as a typical cancer-testis antigen. Such antigens have been recognized as a group of highly attractive targets for cancer therapy (Suda T. et al. Cancer Sci 2007; 98: 1803-8, Mizukami Y. et al. Cancer Sci 2008; 99: 1448-54.). Therefore, selective inhibition of ECT2 enzymatic activity by small molecule compounds is a useful therapeutic strategy against cancer with a minimal risk of adverse events. Moreover, ECT2 oncoantigen is also useful for screening of HLA-restricted epitope peptides for cancer vaccine that can induce specific immune responses by cytotoxic T cells against cancer cells with ECT2 expression. Since the data presented here show that ECT2 has fundamental functions that are responsible for cancer cell survival, vaccination with the peptides from this protein will reduce the risk of the emergence of immune escape variant tumors that have lost their antigen expression.

In conclusion, these data provide the basis for designing new anti-cancer drugs to specifically target the oncogenic activity of ECT2 for the treatment of cancer patients. ECT2 overexpression in resected specimens is also a useful index for application of adjuvant therapy to the lung and esophageal patients who are likely to have poor clinical outcome.

### INDUSTRIAL APPLICABILITY

The gene-expression analysis of cancers described herein, using the combination of laser-capture dissection and genome-wide cDNA microarray, has identified specific genes as targets for cancer prevention and therapy. Based on the expression of a subset of these differentially expressed genes, the present invention provides molecular diagnostic markers for identifying and detecting cancers as well as assessing the prognosis.

The methods described herein are also useful for the identification of additional molecular targets for prevention, diagnosis, and treatment of cancers. The data provided herein add to a comprehensive understanding of cancers, facilitate development of novel diagnostic strategies, and provide molecular targets for therapeutic drugs and preventative agents. Such information contributes to a more profound under-standing of tumorigenesis, and provide novel strategies for diagnosis, treatment, and ultimately prevention of cancers.

All patents, patent applications, and publications, including GenBank accessions, cited herein are incorporated by reference in their entirety.

Furthermore, while the invention has been described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention. Thus, the invention is intended to be defined not by the above description, but by the following claims and their equivalents.

1. A method for assessing the prognosis of a patient with lung or esophageal cancer, which method comprises the steps of:
a) detecting the expression level of the ECT2 gene in a patient-derived biological sample;
b) comparing the detected expression level to a control level; and
c) determining the prognosis of the patient based on the comparison of (b).

2. The method of claim 1, wherein the lung cancer is NSCLC.

3. The method of claim 1, wherein the esophageal cancer is ESCC.

4. The method of claim 1, wherein the control level corresponds to a good prognosis control level and an increase of the expression level as compared to the control level is determined as poor prognosis.

5. The method of claim 4, wherein the ECT2 expression level is at least 10% greater than said control level.

6. The method of claim 1, wherein said method further comprises the step of determining the expression level of other lung or esophageal cancer-associated genes.

7. The method of claim 1, wherein said expression level is determined by a method selected from the group consisting of:
a) detecting mRNA of the ECT2 gene;
b) detecting the ECT2 protein; and
c) detecting the biological activity of the ECT2 protein.

8. The method of claim 1, wherein said expression level is determined by detecting hybridization of a probe to a gene transcript of the ECT2 gene.

9. The method of claim 8, wherein the hybridization step is carried out on a DNA array.

10. The method of claim 1, wherein said expression level is determined by detecting the binding of an antibody against the ECT2 protein.

11. The method of claim 1, wherein said biological sample comprises sputum or blood.

12-35. (canceled)

36. A method of treating or preventing lung or esophageal cancer in a subject comprising administering to said subject a pharmaceutically effective amount of a double-stranded molecule inhibiting the expression of ECT2 gene in a cell, wherein said double-stranded molecule comprises a sense strand and an antisense strand complementary thereto, hybridized to each other to form the double-stranded molecule and targets to a nucleotide sequence selected from the group consisting of SEQ ID NOs: 1 or 2.


SEQUENCE LISTING (12 sequences)

SEQ ID NO: 1 - RNA, 19 nt, Artificial Sequence
  An artificially synthesized siRNA sequence
gauaaaggaugaucuugaa

SEQ ID NO: 2 - RNA, 19 nt, Artificial Sequence
  An artificially synthesized siRNA sequense
cagaggagauuaagacuau

SEQ ID NO: 3 - DNA, 23 bp, Artificial Sequence
  An artificially synthesyzed primer sequence
gcgttttcaagatctagcatgtg

SEQ ID NO: 4 - DNA, 22 bp, Artificial Sequence
  An artificially synthesyzed primer sequence
caattttcccatggtcttatcc

SEQ ID NO: 5 - DNA, 21 bp, Artificial Sequence
  An artificially synthesyzed primer sequence
gaggtgatagcattgctttcg

SEQ ID NO: 6 - DNA, 21 bp, Artificial Sequence
  An artificially synthesyzed primer sequence
caagtcagtgtacaggtaagc

SEQ ID NO: 7 - DNA, 20 bp, Artificial Sequence
  An artificially synthesyzed primer sequence
tggtgaaagctggaaggaag

SEQ ID NO: 8 - protein, 181 aa, Homo sapiens
PLSQIKKVLDIRETEDCHNAFALLVRPPTEQANVLLSFQMTSDELPKENWLKMLCRHVANTICKADAENLIYTADPESFEVNTKDMDSTLSRASRAIKKTSKKVTRAFSFSKTPKRALRRALMTSHGSVEGRSPSSNDKHVMSRLSSTSSLAGIPSPSLVSLPSFFERRSHTLSRSTTHLI

SEQ ID NO: 9 - RNA, 19 nt, Artificial Sequence
  An artificially synthesyzed siRNA sequence
cguacgcggaauacuucga

SEQ ID NO: 10 - RNA, 19 nt, Artificial Sequence
  An artificially synthesyzed siRNA sequence
gcgcgcuuuguaggauucg

SEQ ID NO: 11 - DNA, 3916 bp, Homo sapiens
  CDS (29)..(2680)
agagtgctgatttagaagaatacaaatcatggctgaaaatagtgtattaacatccactactgggaggactagcttggcagactcttccatttttgattctaaagttactgagatttccaaggaaaacttacttattggatctacttcatatgtagaagaagagatgcctcagattgaaacaagagtgatattggttcaagaagctggaaaacaagaagaacttataaaagccttaaaggacattaaagtgggctttgtaaagatggagtcagtggaagaatttgaaggtttggattctccggaatttgaaaatgtatttgtagtcacggactttcaggattctgtctttaatgacctctacaaggctgattgtagagttattggaccaccagttgtattaaattgttcacaaaaaggagagcctttgccattttcatgtcgcccgttgtattgtacaagtatgatgaatctagtactatgctttactggatttaggaaaaaagaagaactagtcaggttggtgacattggtccatcacatgggtggagttattcgaaaagactttaattcaaaagttacacatttggtggcaaattgtacacaaggagaaaaattcagggttgctgtgagtctaggtactccaattatgaagccagaatggatttataaagcttgggaaaggcggaatgaacaggatttctatgcagcagttgatgactttagaaatgaatttaaagttcctccatttcaagattgtattttaagtttcctgggattttcagatgaagagaaaaccaatatggaagaaatgactgaaatgcaaggaggtaaatatttaccgcttggagatgaaagatgcactcaccttgtagttgaagagaatatagtaaaagatcttccctttgaaccttcaaagaaactttatgttgtcaagcaagagtggttctggggaagcattcaaatggatgcccgagctggagaaactatgtatttatatgaaaaggcaaatactcctgagctcaagaaatcagtgtcaatgctttctctaaatacccctaacagcaatcgcaaacgacgtcgtttaaaagaaacacttgctcagctttcaagagagacagacgtgtcaccatttccaccccgtaagcgcccatcagctgagcattccctttccatagggtcactcctagatatctccaacacaccagagtctagcattaactatggagacaccccaaagtcttgtactaagtcttctaaaagctccactccagttccttcaaagcagtcagcaaggtggcaagttgcaaaagagctttatcaaactgaaagtaattatgttaatatattggcaacaattattcagttatttcaagtaccattggaagaggaaggacaacgtggtggacctatccttgcaccagaggagattaagactatttttggtagcatcccagatatctttgatgtacacactaagataaaggatgatcttgaagaccttatagttaattgggatgagagcaaaagcattggtgacatttttctgaaatattcaaaagatttggtaaaaacctaccctccctttgtaaacttctttgaaatgagcaaggaaacaattattaaatgtgaaaaacagaaaccaagatttcatgcttttctcaagataaaccaagcaaaaccagaatgtggacggcagagccttgttgaacttcttatccgaccagtacagaggttacccagtgttgcattacttttaaatgatcttaagaagcatacagctgatgaaaatccagacaaaagcactttagaaaaagctattggatcactgaaggaagtaatgacgcatattaatgaggataagagaaaaacagaagctcaaaagcaaatttttgatgttgtttatgaagtagatggatgcccagctaatcttttatcttctcaccgaagcttagtacagcgggttgaaacaatttctctaggtgagcacccctgtgacagaggagaacaagtaactctcttcctcttcaatgattgcctagagatagcaagaaaacggcacaaggttattggcacttttaggagtcctcatggccaaacccgacccccagcttctcttaagcatattcacctaatgcctctttctcagattaagaaggtattggacataagagagacagaagattgccataatgcttttgccttgcttgtgaggccaccaacagagcaggcaaatgtgctactcagtttccagatgacatcagatgaacttccaaaagaaaactggctaaagatgctgtgtcgacatgtagctaacaccatttgtaaagcagatgctgagaatcttatttatactgctgatccagaatcctttgaagtaaatacaaaagatatggacagtacattgagtagagcatcaagagcaataaaaaagacttcaaaaaaggttacaagagcattctctttctccaaaactccaaaaagagctcttcgaagggctcttatgacatcccacggctcagtggagggaagaagtccttccagcaatgataagcatgtaatgagtcgtctttctagcacatcatcattagcaggtatcccttctccctcccttgtcagccttccttccttctttgaaaggagaagtcatacgttaagtagatctacaactcatttgatatgaagcgttaccaaaatcttaaattatagaaatgtatagacacctcatactcaaataagaaactgacttaaatggtacttgtaattagcacttggtgaaagctggaaggaagataaataacactaaactatgctatttgatttttcttcttgaaagagtaaggtttacctgttacattttcaagttaattcatgtaaaaaatgatagtgattttgatgtaatttatctcttgtttgaatctgtcattcaaaggccaataatttaagttgctatcagctgatattagtagctttgcaaccctgatagagtaaataaattttatgggcgggtgccaaatactgctgtgaatctatttgtatagtatccatgaatgaatttatggaaatagatatttgtgcagctcaatttatgcagagattaaatgacatcataatactggatgaaaacttgcatagaattctgattaaatagtgggtctgtttcacatgtgcagtttgaagtatttaaataaccactcctttcacagtttattttcttctcaagcgttttcaagatctagcatgtggattttaaaagatttgccctcattaacaagaataacatttaaaggagattgtttcaaaatatttttgcaaattgagataaggacagaaagattgagaaacattgtatattttgcaaaaacaagatgtttgtagctgtttcagagagagtacggtatatttatggtaattttatccactagcaaatcttgatttagtttgatagtgtgtggaattttattttgaaggataagaccatgggaaaattgtggtaaagactgtttgtacccttcatgaaataattctgaagttgccatcagttttactaatcttctgtgaaatgcatagatatgcgcatgttcaactttttattgtggtcttataattaaatgtaaaattgaaaattcatttgctgtttcaaagtgtgatatctttcacaatagcctttttatagtcagtaattcagaataatcaagttcatatggataaatgcatttttatttcctatttctttagggagtgctacaaatgtttgtcacttaaatttcaagtttctgttttaatagttaactgactatagattgttttctatgccatgtatgtgccacttctgagagtagtaaatgactctttgctacattttaaaagcaattgtattagtaagaactttgtaaataaatacctaaaacccaagtgt

SEQ ID NO: 12 - protein, 883 aa, Homo sapiens
MAENSVLTSTTGRTSLADSSIFDSKVTEISKENLLIGSTSYVEEEMPQIETRVILVQEAGKQEELIKALKDIKVGFVKMESVEEFEGLDSPEFENVFVVTDFQDSVFNDLYKADCRVIGPPVVLNCSQKGEPLPFSCRPLYCTSMMNLVLCFTGFRKKEELVRLVTLVHHMGGVIRKDFNSKVTHLVANCTQGEKFRVAVSLGTPIMKPEWIYKAWERRNEQDFYAAVDDFRNEFKVPPFQDCILSFLGFSDEEKTNMEEMTEMQGGKYLPLGDERCTHLVVEENIVKDLPFEPSKKLYVVKQEWFWGSIQMDARAGETMYLYEKANTPELKKSVSMLSLNTPNSNRKRRRLKETLAQLSRETDVSPFPPRKRPSAEHSLSIGSLLDISNTPESSINYGDTPKSCTKSSKSSTPVPSKQSARWQVAKELYQTESNYVNILATIIQLFQVPLEEEGQRGGPILAPEEIKTIFGSIPDIFDVHTKIKDDLEDLIVNWDESKSIGDIFLKYSKDLVKTYPPFVNFFEMSKETIIKCEKQKPRFHAFLKINQAKPECGRQSLVELLIRPVQRLPSVALLLNDLKKHTADENPDKSTLEKAIGSLKEVMTHINEDKRKTEAQKQIFDVVYEVDGCPANLLSSHRSLVQRVETISLGEHPCDRGEQVTLFLFNDCLEIARKRHKVIGTFRSPHGQTRPPASLKHIHLMPLSQIKKVLDIRETEDCHNAFALLVRPPTEQANVLLSFQMTSDELPKENWLKMLCRHVANTICKADAENLIYTADPESFEVNTKDMDSTLSRASRAIKKTSKKVTRAFSFSKTPKRALRRALMTSHGSVEGRSPSSNDKHVMSRLSSTSSLAGIPSPSLVSLPSFFERRSHTLSRSTTHLI